SEQUENCE LISTING (13 sequences)

SEQ ID NO: 1 - DNA, 1335 bp, Artificial Sequence
  Synthetic exemplary nucleic acid sequence for NLS-pMag-CreC
cccaagaagaagaggaaagtcggcggacatactctttatgcccccggtggatatgacattatgggatatctgaggcagatcaggaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaaggtaccaacaggaaatggttccctgctgaacctgaggatgtgagggactacctcctgtacctgcaagccagaggcctggctgtgaagaccatccaacagcacctgggccagctcaacatgctgcacaggagatctggcctgcctcgcccttctgactccaatgctgtgtccctggtgatgaggagaatcagaaaggagaatgtggatgctggggagagagccaagcaggccctggcctttgaacgcactgactttgaccaagtcagatccctgatggagaactctgacagatgccaggacatcaggaacctggccttcctgggcattgcctacaacaccctgctgcgcattgccgaaattgccagaatcagagtgaaggacatctcccgcaccgatggtgggagaatgctgatccacattggcaggaccaagaccctggtgtccacagctggtgtggagaaggccctgtccctgggggttaccaagctggtggagagatggatctctgtgtctggtgtggctgatgaccccaacaactacctgttctgccgggtcagaaagaatggtgtggctgccccttctgccacctcccaactgtccacccgggccctggaagggatctttgaggccacccaccgcctgatctatggtgccaaggatgactctgggcagagatacctggcctggtctggccactctgccagagtgggtgctgccagggacatggccagggctggtgtgtccatccctgaaatcatgcaggctggtggctggaccaatgtgaacattgtgatgaactacatcagaaacctggactctgagactggggccatggtgaggctgctcgaggatggggac

SEQ ID NO: 2 - DNA, 939 bp, Artificial Sequence
  Synthetic exemplary nucleic acid sequence for ERT2
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccaca

SEQ ID NO: 3 - DNA, 579 bp, Artificial Sequence
  Synthetic exemplary nucleic acid sequence for CreN-nMag
acgagtgatgaggttcgcaagaacctgatggacatgttcagggatcgccaggcgttttctgagcatacctggaaaatgcttctgtccgtttgccggtcgtgggcggcatggtgcaagttgaatggtacccatactctttatgcccccggtggatatgacattatgggatatctggaccagatcggcaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaa

SEQ ID NO: 4 - DNA, 1575 bp, Artificial Sequence
  Synthetic exemplary nucleic acid sequence ERT2 linked to CreN-nMag
atggctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagtgaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggaggcggcggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacagctgtcgacaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaatggtacccatactctttatgcccccggtggatatgacattatgggatatctggaccagatcggcaaccggccaaacccgcaggtggaactgggccccgtggatacatcctgcgccttgattctttgtgacctgaaacagaaagacaccccgatagtttacgcgagtgaagccttcctctacatgacaggttacagcaacgcagaggtgctgggccggaattgccggtttctgcaaagccctgacggcatggtgaagcccaagagcacccggaagtacgtggatagtaacacaatcaatactatgcgcaaggcaatcgacaggaatgccgaggtgcaggttgaagtagtcaattttaaaaagaatggacagcgatttgttaatttcctgactatgatacctgttagggacgaaacaggcgagtatcgatactctatgggattccagtgcgaaacagaa

SEQ ID NO: 5 - DNA, 34 bp, Artificial Sequence
  Synthetic exemplary nucleic acid sequence for exemplary lox P site
ataacttcgtatagcatacattatacgaagttat

SEQ ID NO: 6 - DNA, 57 bp, Artificial Sequence
  Synthetic linker
gctgtcgacaatttactgaccgtacaccaaaatttgcctgcattaccggtcgatgca

SEQ ID NO: 7 - DNA, 1179 bp, Artificial Sequence
  Synthetic exemplary nucleic acid sequence for exemplary EF1alpha promoter
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 8 - DNA, 1089 bp, Artificial Sequence
  Synthetic exemplary nucleic acid sequence for exemplary myc-alpha-CD38-Receptor
atggagtttggactgagctggttgttcctcgtagctattctgaagggcgttcagtgctctgagcaaaagttgatctcagaagaggacctggacatccagttgacccagagtagctcctccttctctgtgagcctgggagatcgggtgactattacctgtaaagcgtctgaagatatttacaaccgcctggcgtggtaccagcagaaaccaggaaacgccccaagacttctcatatctggagccactagccttgaaactggcgtaccctcccgattttccgggtccggaagtggcaaagattataccctctccattacctctcttcagacggaggacgtggcaacatactattgtcaacagtactggtctacacctacctttggtgggggaaccaagctggaaatcaagagggccgatgccgctcccactgtttcaatcttcccaccctctggcggcggcgggagcggcggtggtggcagcgggggtggtgggtccggaggtggggggtcacaggtccagctgaagcaaagcggccctagtctcgtccagccctcacaaagactgtccatcacctgtaccgtcagcggcttttctctgatcagctatggagtccattgggtgaggcaatctcctggaaagggactggaatggctgggggtgatctggaggggcgggtccaccgattacaacgccgcattcatgtcacgcctgtccatcactaaagacaactccaaatctcaagtgttctttaaaatgaactccctgcaggctgatgacaccgctatctacttctgtgcaaaaacgctgataactacgggttacgccatggattactggggccagggtacctccgtcaccgttagctctgccgcagctatcgaggtcatgtacccgccaccctatctcgataacgaaaaatccaacgggacaatcatacacgtaaagggcaagcatttgtgcccctcccctttgttcccagggccgtcaaaaccattctgggtcttggtagtcgtaggaggtgtgcttgcttgttactcactgttggttactgttgcgttcataatcttttgggtgcggtcaaaacgctcc

SEQ ID NO: 9 - DNA, 2346 bp, Artificial Sequence
  Synthetic exemplary nucleic acid sequence for exemplary alpha-CD19CAR-EGFP
atggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgctttactgcaaccacagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtggaagtggcggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 10 - DNA, 4807 bp, Artificial Sequence
  Synthetic exemplary nucleic acid sequence for exemplary CAR Reporter (EF1alpha promoter-LoxP-myc-alpha-CD38-Receptor-LoxP-alpha-CD19CAR-EGFP)
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattgatccttcgaactagtttctgcagtcgaccggaattataacttcgtatagcatacattatacgaagttataattctgcagatatccagcacagtggcggccaccatggagtttggactgagctggttgttcctcgtagctattctgaagggcgttcagtgctctgagcaaaagttgatctcagaagaggacctggacatccagttgacccagagtagctcctccttctctgtgagcctgggagatcgggtgactattacctgtaaagcgtctgaagatatttacaaccgcctggcgtggtaccagcagaaaccaggaaacgccccaagacttctcatatctggagccactagccttgaaactggcgtaccctcccgattttccgggtccggaagtggcaaagattataccctctccattacctctcttcagacggaggacgtggcaacatactattgtcaacagtactggtctacacctacctttggtgggggaaccaagctggaaatcaagagggccgatgccgctcccactgtttcaatcttcccaccctctggcggcggcgggagcggcggtggtggcagcgggggtggtgggtccggaggtggggggtcacaggtccagctgaagcaaagcggccctagtctcgtccagccctcacaaagactgtccatcacctgtaccgtcagcggcttttctctgatcagctatggagtccattgggtgaggcaatctcctggaaagggactggaatggctgggggtgatctggaggggcgggtccaccgattacaacgccgcattcatgtcacgcctgtccatcactaaagacaactccaaatctcaagtgttctttaaaatgaactccctgcaggctgatgacaccgctatctacttctgtgcaaaaacgctgataactacgggttacgccatggattactggggccagggtacctccgtcaccgttagctctgccgcagctatcgaggtcatgtacccgccaccctatctcgataacgaaaaatccaacgggacaatcatacacgtaaagggcaagcatttgtgcccctcccctttgttcccagggccgtcaaaaccattctgggtcttggtagtcgtaggaggtgtgcttgcttgttactcactgttggttactgttgcgttcataatcttttgggtgcggtcaaaacgctcctgataagctagctgaataaggccgctcgaataacttcgtatagcatacattatacgaagttatgtttcgggaattcgatggctctcccagtgactgccctactgcttcccctagcgcttctcctgcatgcagaggtgaagctgcagcagtctggggctgagctggtgaggcctgggtcctcagtgaagatttcctgcaaggcttctggctatgcattcagtagctactggatgaactgggtgaagcagaggcctggacagggtcttgagtggattggacagatttatcctggagatggtgatactaactacaatggaaagttcaagggtcaagccacactgactgcagacaaatcctccagcacagcctacatgcagctcagcggcctaacatctgaggactctgcggtctatttctgtgcaagaaagaccattagttcggtagtagatttctactttgactactggggccaagggaccacggtcaccgtctcctcaggtggaggtggatcaggtggaggtggatctggtggaggtggatctgacattgagctcacccagtctccaaaattcatgtccacatcagtaggagacagggtcagcgtcacctgcaaggccagtcagaatgtgggtactaatgtagcctggtatcaacagaaaccaggacaatctcctaaaccactgatttactcggcaacctaccggaacagtggagtccctgatcgcttcacaggcagtggatctgggacagatttcactctcaccatcactaacgtgcagtctaaagacttggcagactatttctgtcaacaatataacaggtatccgtacacgtccggaggggggaccaagctggagatcaaacgggcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcccgtttctctgttgttaaacggggcagaaagaagctcctgtatatattcaaacaaccatttatgagaccagtacaaactactcaagaggaagatggctgtagctgccgatttccagaagaagaagaaggaggatgtgaactgctttactgcaaccacagagtgaagttcagcaggagcgcagagccccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggtggaagtggcggtaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatag

SEQ ID NO: 11 - DNA, 12 bp, Artificial Sequence
  Synthetic Linker
tgataagctagc

SEQ ID NO: 12 - DNA, 14 bp, Artificial Sequence
  Synthetic Linker
gtttcgggaattcg

SEQ ID NO: 13 - DNA, 17 bp, Artificial Sequence
  Synthetic stop codon cassette sequence
tgaataaggccgctcga

(19) United States
(12) Patent Application Publication
(10) Pub. No.: US 2023/0036060 A1
(43) Pub. Date: Feb. 2, 2023

(54) DUAL-CONTROLLED DRUG AND PHOTOACTIVATABLE SYSTEM FOR SPATIOTEMPORAL CONTROL OF CELL THERAPY

(71) Applicant: THE REGENTS OF THE UNIVERSITY OF CALIFORNIA, Oakland, CA (US)

(72) Inventors: Yingxiao WANG, Oakland, CA (US); Molly E. ALLEN, Oakland, CA (US); Ziliang HUANG, Oakland, CA (US)

(21) Appl. No.: 17/763,900

(22) PCT Filed: Sep. 25, 2020
(86) PCT No.: PCT/US20/52718

§ 371 Date: Mar. 25, 2022

Related U.S. Application Data
(60) Provisional application No. 62/907,279, filed on Sep. 27, 2019.

(51) Int. Cl. C12N 15/63; C12N 9/12

(52) U.S. Cl. CPC C12N 15/635; C12N 9/1241; C07K 2319/09; C07K 2319/32; C12N 2740/15043; C12N 2830/002; C12N 2800/30

(57) ABSTRACT

Provided are compositions, including products of manufacture and kits, and methods, for remotely-controlled and non-invasive manipulation of intracellular nucleic acid expression, genetic processes, function and activity in live cells such as a T cell, a primary T cell, a B cell, a monocyte, a macrophage, a dendritic cell or a natural killercell in vivo, for example, including activating, adding functions or changing or adding specificities for an immune cell, for monitoring physiologic processes, for the correction of pathological processes and for the control of therapeutic outcomes. Provided are tamoxifen-gated photoactivatable split-Cre recombinase optogenetic systems, called TamPA-Cre, that feature high spatiotemporal control to control or alter cell activities in vivo, for example, to limit the activity of a Chimeric Antigen Receptor (CAR)-expressing cell such as an immune cell and its activity at a tumor site for immunotherapy applications.

### RELATED APPLICATIONS

This Patent Convention Treaty (PCT) International Application claims the benefit of priority to U.S. Provisional Application Ser. No. (U.S. Ser. No.) 62/907,279 filed Sep. 27, 2019. The aforementioned application is expressly incorporated herein by reference in its entirety and for all purposes.

### STATEMENT AS TO FEDERALLY SPONSORED RESEARCH

This invention was made with government support under GM126016; GM125379; CA204704; CA209629; and HL121365, awarded by the National Institutes of Health (NIH), and HL105373, awarded by NHLBI. The government has certain rights in the invention.

### REFERENCE TO SEQUENCE LISTING SUBMITTED VIA EFS-WEB

This application includes an electronically submitted sequence listing in .txt format. The .txt file contains a sequence listing entitled “0321.138791PCT_ST25” created on Sep. 25, 2020 and is 21,250 bytes in size. The sequence listing contained in this .txt file is part of the specification and is hereby incorporated by reference herein in its entirety.

### TECHNICAL FIELD

This invention generally relates to improved and focused systems for expressing exogenous nucleic acids in vivo, including expression of anti-cancer chimeric T cell or NK cell receptors. In alternative embodiments, provided are compositions, including products of manufacture and kits, and methods, for remotely-controlled and non-invasive manipulation of intracellular nucleic acid expression, genetic processes, function and activity in live cells such as a T cell, a primary T cell, a B cell, a monocyte, a macrophage, a dendritic cell or a natural killercell in vivo, for example, activating, adding functions or changing or adding specificities for immune cells, for monitoring physiologic processes, for the correction of pathological processes and for the control of therapeutic outcomes. In alternative embodiments, provided are tamoxifen-gated photoactivatable split-Cre recombinase optogenetic systems, called TamPA-Cre, that feature high spatiotemporal control to control or alter cell activities in vivo, for example, to limit the activity of a Chimeric Antigen Receptor (CAR)-expressing cell such as a T cell, a primary T cell, a B cell, a monocyte, a macrophage, a dendritic cell or a natural killercell, and its activity at a tumor site for immunotherapy applications. In alternative embodiments, exemplary optogenetic systems are provided herein allow a deep penetration of stimulation and manipulation in vivo at centimeter-level depth with high spatiotemporal precision.

### BACKGROUND

Artificial T cell receptors (also known as chimeric T cell receptors, chimeric immunoreceptors, chimeric antigen receptors (CARs)) are engineered receptors, which graft a desired specificity onto an immune effector cell such as a T cell. CAR T cell therapy is becoming a paradigm-shifting therapeutic approach for cancer treatment, particularly with the benefit of resulted central memory T cells capable of lasting for months to years in suppressing the cancer relapse. In this therapy, T cells are removed from a cancer patient and modified to express CARs that target the cancer. These modified T cells, which can recognize and kill the patient's cancer cells, are re-introduced into the patient.

However, major challenges remain before CAR-based immunotherapy can become widely adopted. For instance, the non-specific targeting of the CAR-T cells against normal/nonmalignant tissues (on-target but off-tumor toxicities) can be life-threatening. In fact, off- tumor toxicities against the lung, gray matter in the brain, and cardiac muscles, have caused multiple cases of deaths. While synthetic biology and genetic circuits have been used in attempts to address this issue, there is an urgent need for high-precision control of CAR-T cells to confine the activation in tissue space.

In immunotherapy, the expression of engineered CAR on the cell surface enables T cells to recognize specific antigens on the target cell. This triggers T cell activation and can eventually lead to the elimination of target cells. Clinical trials involving anti-CD19 CAR T cells against B-cell malignancies have shown promising results, demonstrating the therapeutic effects of CAR T cells in cancer treatment. However, the perfusion of constitutively activated CAR T cells into patients may have lethal consequences due to the induced cytokine storm and ‘on-target, off tumor’ toxicity. Therefore, researchers are actively seeking control over the timing and location of the activation of the perfused CAR T cells. Given the complexity of immune system and the largely overlapping functions of its molecular regulators, it is a daunting challenge to manipulate immune system at global levels with predictable net outcomes.

### SUMMARY

In alternative embodiments, provided are methods for remotely-controlling and non-invasively manipulating expression of an exogenous nucleic acid in a cell, or an immune cell, and optionally modifying or adding a target capability or a function to the cell, or immune cell,

wherein optionally the immune cell is a T cell, a primary T cell, a B cell, a monocyte, a macrophage, a dendritic cell or a natural killercell,

wherein optionally the exogenous nucleic acid is contained in a vector or expression cassette,

and optionally the exogenous nucleic acid comprises a nucleic acid encoding and capable of expressing a protein, and optionally the protein is a therapeutic protein, or a transcriptional or translational regulatory protein, or a receptor, or a recombinant or an artificial T cell receptor (also known as a chimeric T cell receptor, a chimeric immunoreceptor, a chimeric antigen receptor (CAR), an antibody, a single chain antibody, or a single-domain antibody (also known as sdAb or nanobody) or an antibody fragment consisting of a single monomeric variable antibody domain,

the method comprising:

(a) inserting or expressing in a recombinantly engineered cell:
(i) a tamoxifen-gated photoactivatable split-Cre recombinase (TamPA-Cre) optogenetic system comprising:
   (1) a nucleic acid encoding a cytosol-localizing mutant estrogen receptor ligand binding domain ERT2 fused or linked to a N-terminal half of split Cre(2-59aa)-nMag (CreN-nMag) encoding segment, and,
   (2) a nucleic acid encoding an NLS-pMag-CreC protein,
   wherein an expressed NLS-pMag-CreC protein is nucleus-localized and the ERT2-CreN-nMag protein is cytosolically localized,
wherein optionally the recombinantly engineered cell is an immune cell or comprises a plurality of cells or immune cells, and
(ii) a floxed exogenous nucleic acid (floxing refers to the sandwiching of a DNA sequence (which is then said to be foxed) between two lox sites (wherein optionally the lox sites is a lox P site, a lox H site, a lox 511 site, a lox 5171 site, a lox 66 site, a lox 71 site, or equivalent lox sites) also referred to as “flanking/flanked by LoxP”) operatively linked to a transcriptional regulatory element, optionally a constitutive or an inducible promoter, whose expression can be activated by an active Cre-lox site recombination event,

wherein optionally the Cre-lox sites is a Cre-lox P site, a Cre-lox H site, a Cre-lox 511 site, a Cre-lox 5171 site, a Cre-lox 66 site, a Cre-lox 71 site, or equivalent Cre-lox sites;

wherein optionally the constitutive promoter comprises an EF-1 alpha, PGK, CMV, CAG, SFFV, SV40 or equivalent constitutive promoter,

wherein optionally the TamPA-Cre optogenetic system is stably integrated into the genome of the cell, or optionally is episomally expressed or is contained in a non-integrated vector in the cell,

wherein optionally the tamoxifen-gated photoactivatable split-Cre recombinase (TamPA-Cre) optogenetic system and/or the floxed exogenous nucleic acid is contained in a lentivirus vector;

(b) administering or contacting the cell with tamoxifen or 4-hydroxytamoxifen (4-OHT), wherein a tamoxifen metabolite 4-hydroxytamoxifen (4-OHT) binds with the ERT2-CreN-nMag cytosolically localized protein to drive its nuclear localization to prime TamPA-Cre; and

(c) exposing the cell to blue light to drive nMag-pMag heterodimerization, which restores active TamPA-Cre recombinase activity within the cell nucleus, thereby allowing expression of the foxed exogenous nucleic acid.

In alternative embodiments of methods as provided herein:

an exemplary nucleic acid sequence for NLS-pMag-CreC is:

(SEQ ID NO: 1)
CCCAAGAAGAAGAGGAAAGTCggcggaCATACTCT
TTATGCCCCCGGTGGATATGACATTATGGGATATC
TGAGGCAGATCAGGAACCGGCCAAACCCGCAGGTG
GAACTGGGCCCCGTGGATACATCCTGCGCCTTGAT
TCTTTGTGACCTGAAACAGAAAGACACCCCGATAG
TTTACGCGAGTGAAGCCTTCCTCTACATGACAGGT
TACAGCAACGCAGAGGTGCTGGGCCGGAATTGCCG
GTTTCTGCAAAGCCCTGACGGCATGGTGAAGCCCA
AGAGCACCCGGAAGTACGTGGATAGTAACACAATC
AATACTATGCGCAAGGCAATCGACAGGAATGCCGA
GGTGCAGGTTGAAGTAGTCAATTTTAAAAAGAATG
GACAGCGATTTGTTAATTTCCTGACTATGATACCT
GTTAGGGACGAAACAGGCGAGTATCGATACTCTAT
GGGATTCCAGTGCGAAACAGAAggtaccAACAGGA
AATGGTTCCCTGCTGAACCTGAGGATGTGAGGGAC
TACCTCCTGTACCTGCAAGCCAGAGGCCTGGCTGT
GAAGACCATCCAACAGCACCTGGGCCAGCTCAACA
TGCTGCACAGGAGATCTGGCCTGCCTCGCCCTTCT
GACTCCAATGCTGTGTCCCTGGTGATGAGGAGAAT
CAGAAAGGAGAATGTGGATGCTGGGGAGAGAGCCA
AGCAGGCCCTGGCCTTTGAACGCACTGACTTTGAC
CAAGTCAGATCCCTGATGGAGAACTCTGACAGATG
CCAGGACATCAGGAACCTGGCCTTCCTGGGCATTG
CCTACAACACCCTGCTGCGCATTGCCGAAATTGCC
AGAATCAGAGTGAAGGACATCTCCCGCACCGATGG
TGGGAGAATGCTGATCCACATTGGCAGGACCAAGA
CCCTGGTGTCCACAGCTGGTGTGGAGAAGGCCCTG
TCCCTGGGGGTTACCAAGCTGGTGGAGAGATGGAT
CTCTGTGTCTGGTGTGGCTGATGACCCCAACAACT
ACCTGTTCTGCCGGGTCAGAAAGAATGGTGTGGCT
GCCCCTTCTGCCACCTCCCAACTGTCCACCCGGGC
CCTGGAAGGGATCTTTGAGGCCACCCACCGCCTGA
TCTATGGTGCCAAGGATGACTCTGGGCAGAGATAC
CTGGCCTGGTCTGGCCACTCTGCCAGAGTGGGTGC
TGCCAGGGACATGGCCAGGGCTGGTGTGTCCATCC
CTGAAATCATGCAGGCTGGTGGCTGGACCAATGTG
AACATTGTGATGAACTACATCAGAAACCTGGACTC
TGAGACTGGGGCCATGGTGAGGCTGCTCGAGGATG
GGGAC

an exemplary nucleic acid sequence for ERT2 is:

(SEQ ID NO: 2)
Atggctggaggacatgagagctgccaacctttggc
caagcccgctcatgatcaaagcgtctaagaagaac
agcctggccttgtccctgacggccgaccagatggt
cagtgccttgttggatgctgagccccccatactct
attccgagtatgatcctaccagacccttcagtgaa
gcttcgatgatgggcttactgaccaacctggcaga
cagggagctggttcacatgatcaactgggcgaaga
gggtgccaggctttgtggatttgaccctccatgat
caggtccaccttctagaatgtgcctggctagagat
cctgatgattggttctcgtctggcgctccatggag
cacccagtaaagctactgtttgctcctaacttgct
cttggacaggaaccagggaaaatgtgtagagggca
tggtggagatcttcgacatgctgctggctacatca
tctcggttccgcatgatgaatctgcagggagagga
gtttgtgtgcctcaaatctattattttgcttaatt
ctggagtgtacacatttctgtccagcaccctgaag
tctctggaagagaaggaccatatccaccgagtcct
ggacaagatcacagacactttgatccacctgatgg
ccaaggcaggcctgaccctgcagcagcagcaccag
cggctggcccagctcctcctcatcctctcccacat
caggcacatgagtaacaaaggcatggagcatctgt
acagcatgaagtgcaagaacgtggtgcccctctat
gacctgctgctggaggcggcggacgcccaccgcct
acatgcgcccactagccgtggaggggcatccgtgg
aggagacggaccaaagccacttggccactgcgggc
tctacttcatcgcattccttgcaaaagttattaca
tcacgggggaggcagagggtttccctgccaca

an exemplary nucleic acid sequence for CreN-nMag is:

(SEQ ID NO: 3)
acgagtgatgaggttcgcaagaacctgatggacatg
ttcagggatcgccaggcgttttctgagcatacctg
gaaatgcttctgtccgtttgccggtcgtgggcggc
atggtgcaagttgaatggtaccCATACTCTTTATG
CCCCCCGGTGGATATGACATTATGGGATATCTGGA
CCAGATCGGCAACCGGCCAAACCCGCAGGTGGAAC
TGGGCCCCGTGGATACATCCTGCGCCTTGATTCTT
TGTGACCTGAAACAGAAAGACACCCCGATAGTTTA
CGCGAGTGAAGCCTTCCTCTACATGACAGGTTACA
GCAGCGCAGAGGTGCTGGGCCGGAATTGCCGGTTT
CTGCAAAGCCCTGACGGCATGGTGAAGCCCAAGAG
CACCCGGAAGTACGTGGATAGTAACACAATCAATA
CTATGCGCAAGGCAATCGACAGGAATGCCGAGGTG
CAGGTTGAAGTAGTCAATTTTAAAAAGAATGGACA
GCGATTTGTTAATTTCCTGACTATGATACCTGTTA
GGGACGAAACAGGCGAGTATCGATACTCTATGGGA
TTCCAGTCCGAAACAGAA

an exemplary nucleic acid sequence ERT2 (blue) linked to CreN-nMag (red) is:

(SEQ ID NO: 4)
atggctggagactgagagagccaacctttggccaa
gcccgctcatgatcaaacgctctctaagaagaaca
gcctggccttgtccctgacggccgaccagatggtc
agtgccttgttggatgctgagccccccatactcta
ttccgagtatgatcctaccagaccttcagtgaagc
ttcgatgttgggcttactgaccaacctggcagaca
gggagcggttcacatgatcaactgggcgaagaggg
tgccaggctttgtggatttgaccctccatgatcag
gtccaccttctagaatgtgcctggctagagatcct
gatgattggtctcgtctggcgctccatggagcacc
cagtgaagctactgtttgctcctaacttgctcttg
gacaggaaccagggaaaatgtgtagagggcatggt
ggagatcttcgacatgctggctacatcatctcggt
tccgcatgatgaatctgcagggagaggagtttgtg
tgcctcaaatctattattttgcttaattctggagt
gtacagatttctgtccagcaccctgaagtctcgga
agagaaggaccatatccaccgagtcctggacaaga
tcacagacactttgatccacctgatggccaaggca
ggcctgaccctgcagcagcagcaccagcggctggc
ccagctcctcctcatcctctcccacatcaggcaca
tgagtaacaaaggcatggagcatctgtacagcatg
aagtgcaagaacgtggtgcccctctatgacctgct
gctggaggcggcggacgcccaccgcctacatgcgc
cctagccgtggaggggcatccgtggaggagacgga
ccaaagccacttggccactgcgggactccttcatc
gcattccttgcaaaagtattacatcacggggaggc
agagggtttccctgccaca_gctgtcgacaattta
ctgaccgtacaccaaaatttgcctgcattaccggt
cgatgca_acgagtgatgaggttcgcaagaacotg
atggacatgttcagggatcgccaggcgttttctga
gcatacctggaaaatgcttctgtccgtttgccggt
cgtgggcggcatggtgcaagttgaatggtaccCAT
ACTCTTTATGCCCCCGGTGGATATGACATTATGGG
ATATCTGGACCAGATCGGCAACCGGCCAAACCCGC
AGGTGGAACTGGGCCCCGTGGATACATCCTGCGCC
TTGATTCTTTGTGACCTGAAACAGAAAGACACCCC
GATAGTTTACGCGAGTGAAGCCTTCCTCTACATGA
CAGGTTACAGCAACGCAGAGGTGCTGGGCCGGAAT
TGCCGGTTTCTGCAAAGCCCTGACGGCATGGTGAA
GCCCAAGAGCACCCGGAAGTACGTGGATAGTAACA
CAATCAATACTATGCGCAAGGCAATCGACAGGAAT
GCCGAGGTGCAGGTTGAAGTAGTCAATTTTAAAAA
GAATGGACAGCGATTTGTTAATTTCCTGACTATGA
TACCTGTTAGGGACGAAACAGGCGAGTATCGATAC
TCTATGGGATTCCAGTGCGAAACAGAA

an exemplary nucleic acid sequence for an exemplary lox P site is:

(SEQ ID NO: 5)
ATAACTTCGTATAGCATACATTATACGAAGTTAT

In alternative embodiments, although the exemplary construct uses the linker:

(SEQ ID NO: 6)
gctgtcgacaatttactgaccgtacacca
aaatttgcctgcattaccggtcgatgca,

any linker known in the art can be used in place of SEQ ID NO:6, there is no specific minimum or maximum length for a linker that can be used in constructs as provided herein, and there is no specific sequence or structural requirement for any linker that can be used in this or any construct as provided herein.

In alternative embodiments of methods as provided herein:

the recombinantly engineered cell is administered in vivo, optionally the recombinantly engineered cell is administered to an individual in need thereof in vivo, and optionally the individual in need thereof is a human or an animal, and optionally the blue light is administered to only a desired area or location in the individual in need thereof, and optionally the desired area or location in the individual in need thereof is a site of a tumor or a growth, and optionally the recombinantly engineered cell is injected into and/or adjacent or approximate to a cancer of a site of a tumor or a growth;

the expressing of the foxed exogenous nucleic acid in the cell adds a function to the cell, or immune cell, or manipulates a physiologic and/or a genetic process in the cell, or immune cell, and optionally when the upregulated nucleic acid is a nucleic acid expressing (encoding) a CAR, a single chain antibody, or a single-domain antibody (also known as sdAb or nanobody) or an antibody fragment consisting of a single monomeric variable antibody domain, thereby adding a new specificity, function or target cell to a cell, an immune cell or a T cell;

the cell is a human cell or a mammalian cell, or is a recombinantly engineered cell engineered to be transplanted or inserted into a tissue, an organ, an organism or an individual, or is or comprises a non-human transgenic animal genetically engineered to contain one or a plurality of recombinantly engineered cells;

the cell or the individual in need thereof is first exposed to or administered tamoxifen followed by being exposed to or administered a continuous or pulsed blue light,

the tamoxifen (optionally GENOX™, TAMIFEN™) is administered to the individual in need thereof by (or formulated appropriately for) oral, intravenous (IV), intramuscular (IM), subcutaneous or topical administration, and optionally the tamoxifen is formulated as tamoxifen citrate (optionally NOLVADEX™ or SOLTAMOX™), and optionally the tamoxifen is formulated as a liquid, a gel or a solid, and optionally the liquid is formulated at about 10 mg/5 mL tamoxifen, and optionally the solid is a pill, a tablet, a geltab, a nanoparticle or a capsule, and optionally each solid formulation comprises about 15.2 mg of tamoxifen citrate which is equivalent to about 10 mg of tamoxifen, or each solid formulation comprises about 30.4 mg of tamoxifen citrate which is equivalent to about 20 mg of tamoxifen, and optionally the nanoparticle is a polylactide-co-glycolide (PLGA) nanoparticle loaded with tamoxifen or tamoxifen citrate,

wherein optionally the cells are exposed (and optionally the cells are exposed in vivo, for example to the individual in need thereof) to between about 400 to 600 nM 4-hydroxytamoxifen (4-OHT), or about 500 nM 4-OHT,

and optionally blue light is applied to the cells between about 2 to 5 hours, or about 3 hours, following an initial exposure to tamoxifen,

and optionally the blue light frequency is about 400 to 500 nM,

and optionally the blue light is applied in a pulsed manner at about 1 second on to about 59 seconds off, or at about 5 seconds on to about 55 seconds off, optionally repeated over a time period of between about 1 hours and 36 hours, or between about 12 hours and 24 hours,

and optionally the blue light is continuously applied to the cells for between about 1 hour and 24 hours, or between about 2 hours and 12 hours;

a chimeric antigen receptor (CAR) is expressed on a T cell surface after exposure of the T cell to tamoxifen followed by blue light, thereby activating the T cell to attack and/or kill a cancerous tissue, a cancer cell or a tumor cell,

wherein optionally the cancerous tissue, cancer cell or tumor cell is a local or skin or mucosal metastatic head/neck cancer, a melanoma, or a skin cancer or a skin growth;

the cell is inside the body of an animal or a human in need thereof, and the recombinantly engineered cell is focused on or approximate to a tumor or a dysplastic or dysfunctional tissue; and/or

the method is used for the manipulation or correction of a pathological process, optionally, for eradicating a tumor or a cancer in an individual in vivo, wherein optionally the individual is a human or an animal.

In alternative embodiments, provided are uses of a genetically engineered cell as engineered for use in a method as provided herein as a medicament.

In alternative embodiments, provided are uses of a genetically engineered cell as engineered for use in a method as provided herein, as a medicament in a remotely-controlled and non-invasive manipulation of a physiologic and/or a genetic process in a cell, or an immune cell, or for the addition of a function or a target specificity to the cell, or immune cell, or plurality of cells or immune cells, or for the manipulation or correction of a pathological process, optionally, for eradicating a tumor or a cancer in an individual in vivo.

In alternative embodiments, provided are genetically engineered cells as engineered for use in a method as provided herein for use as a medicament, or for use as a medicament in a remotely-controlled and non-invasive manipulation of a physiologic and/or a genetic process in a cell, or an immune cell, or for the addition of a function or a target specificity to the cell, or immune cell, or plurality of cells or immune cells, or for the manipulation or correction of a pathological process, optionally, for eradicating a tumor or a cancer in an individual in vivo.

In alternative embodiments, provided are kits or formulations comprising a genetically engineered cell as engineered for use a method as provided herein.

The details of one or more exemplary embodiments as described herein are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

The patent or application file contains at least one drawing executed in color. Copies of this patent or patent application publication with color drawing(s) will be provided by the Office upon request and payment of the necessary fee.

The drawings set forth herein are illustrative of exemplary embodiments provided herein and are not meant to limit the scope of the invention as encompassed by the claims.

FIG. 1A-H schematically illustrate an exemplary TamPA-Cre application and molecular mechanism:

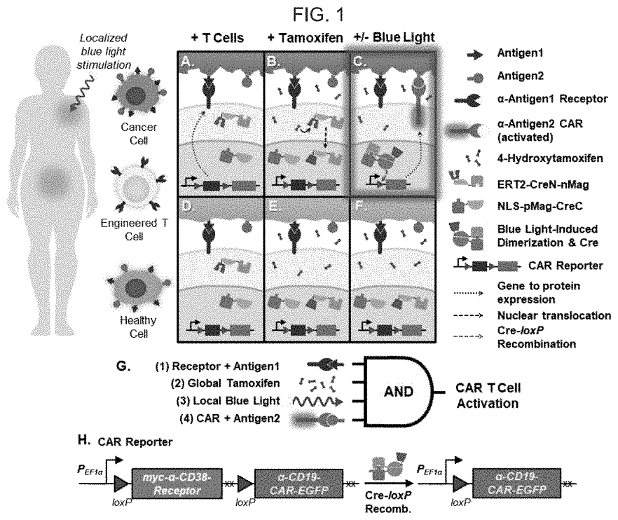

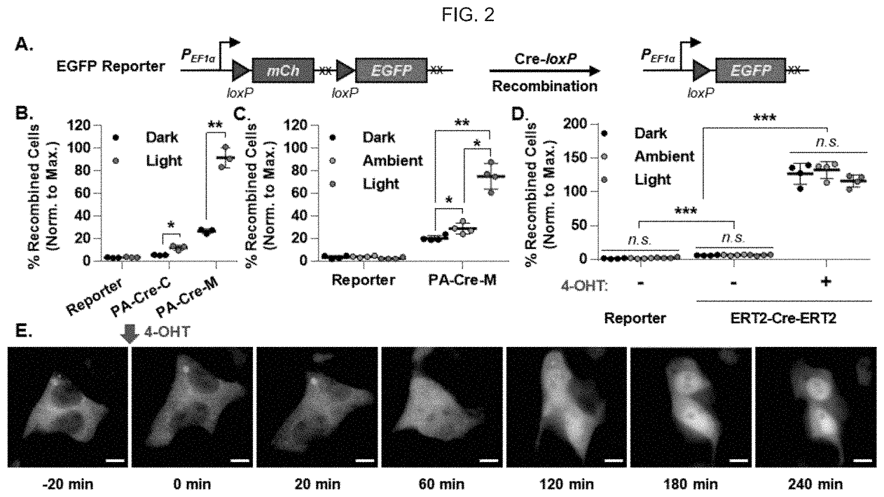

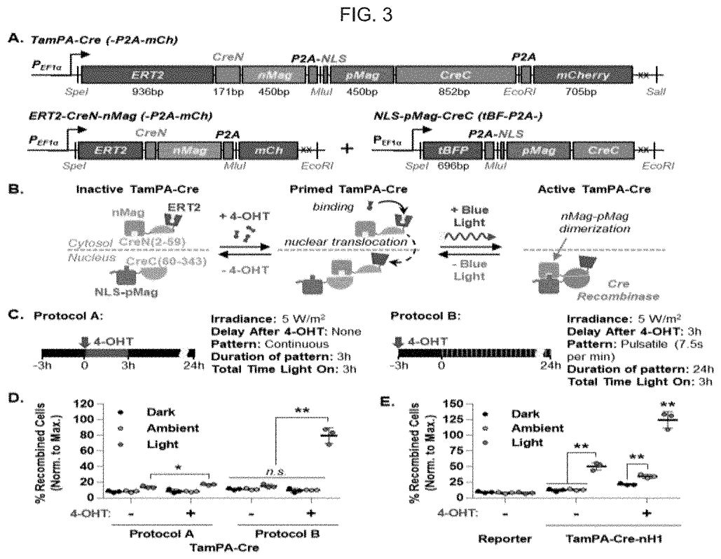

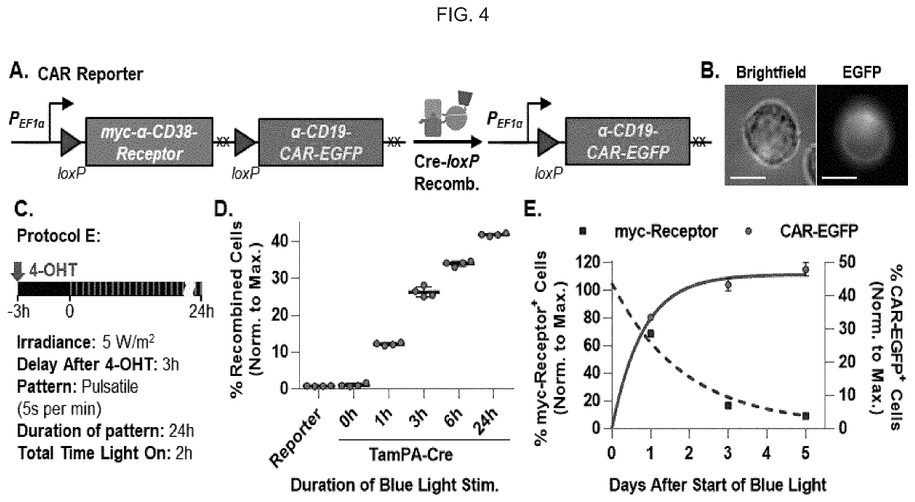

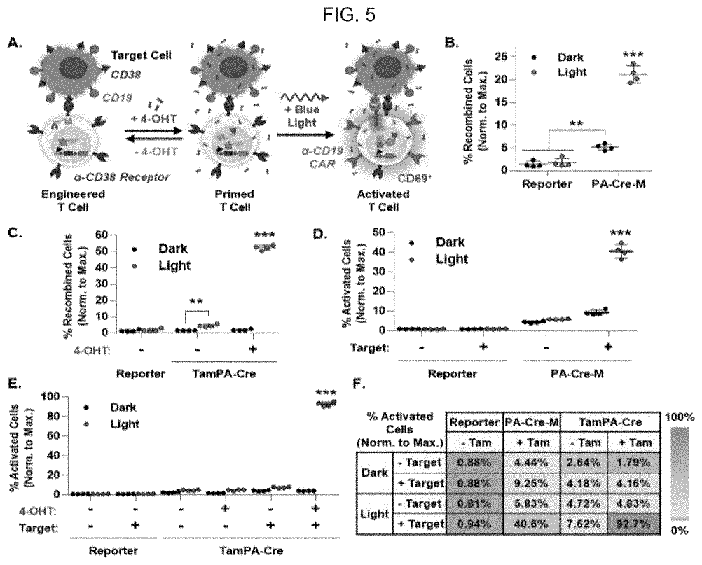

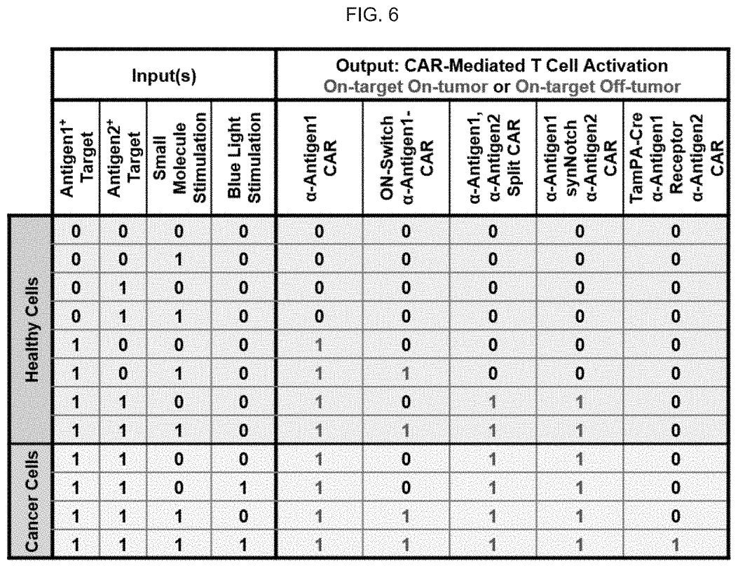

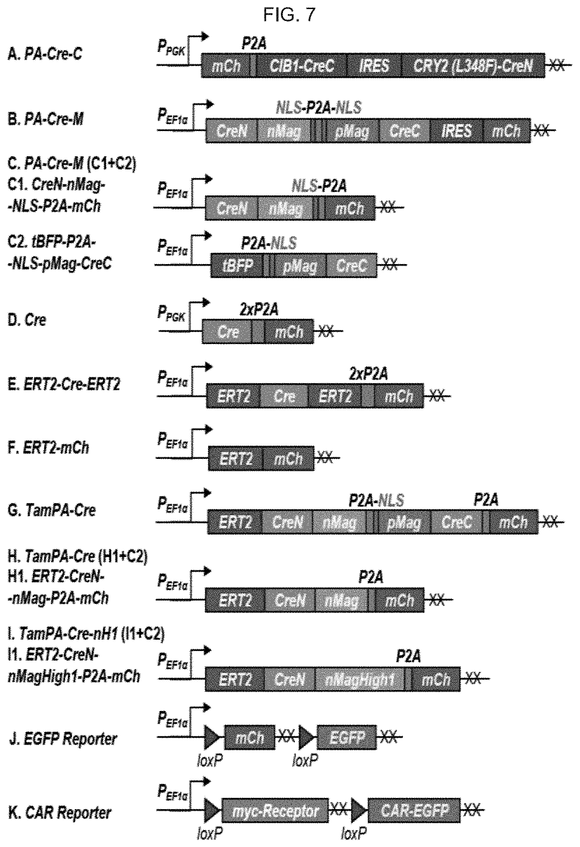

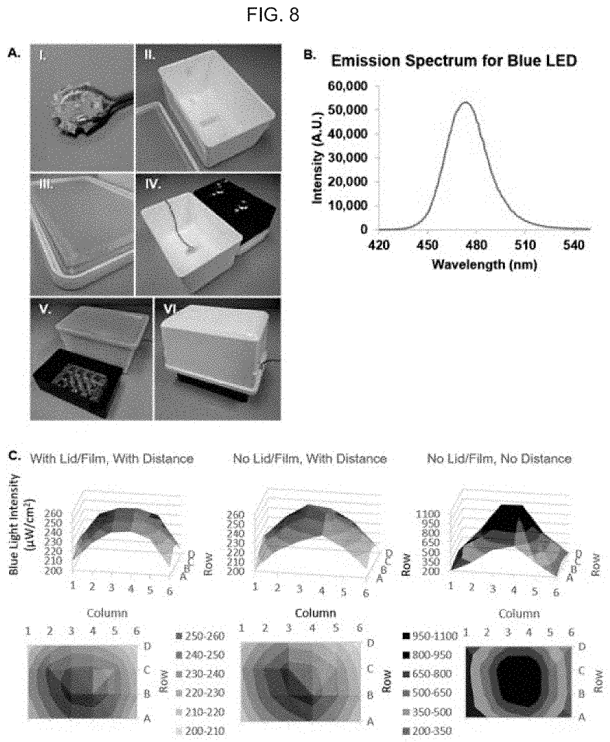

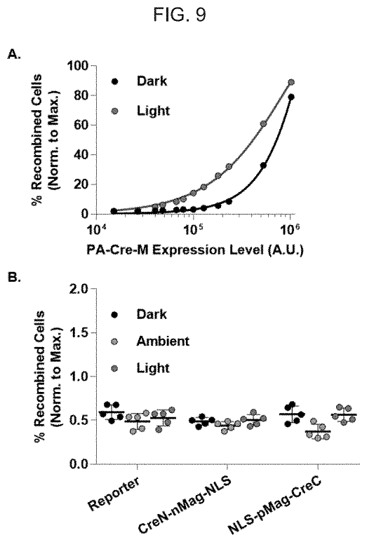

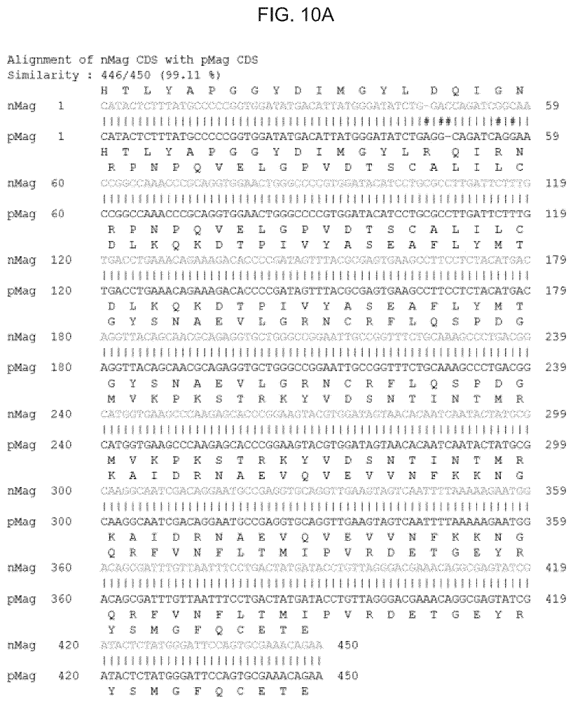

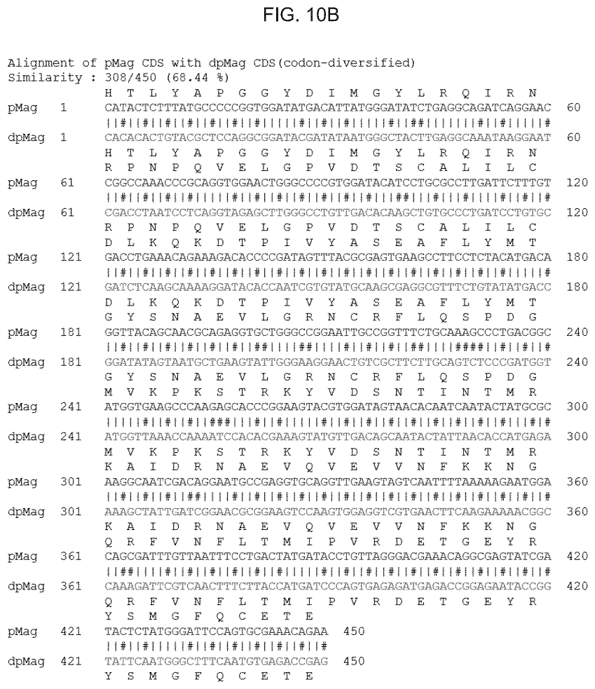

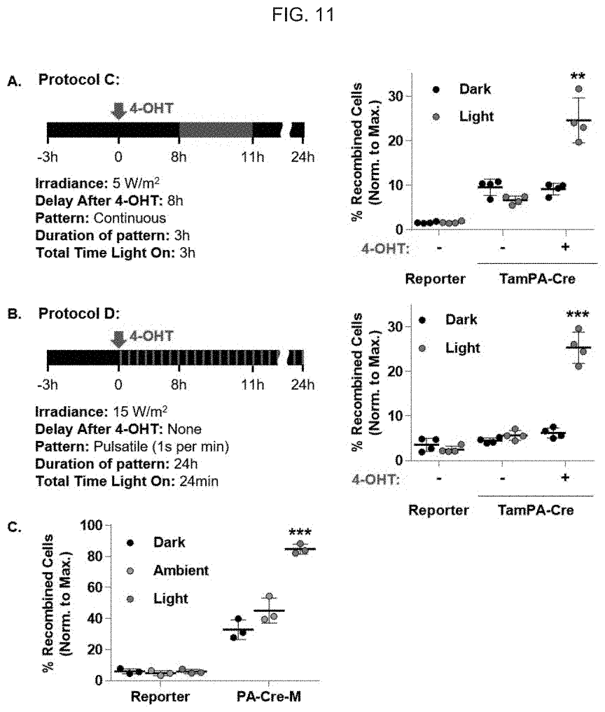

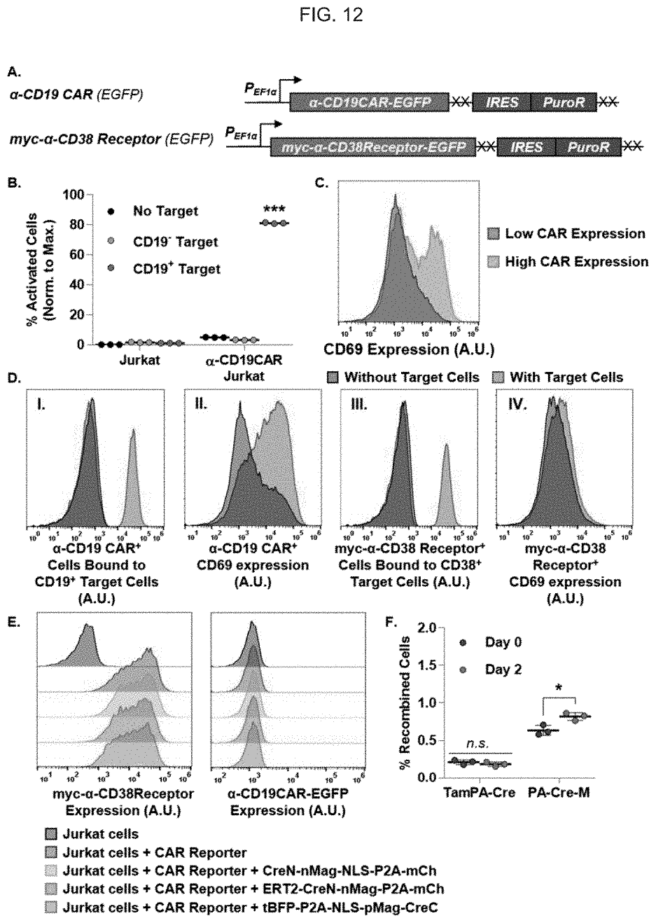

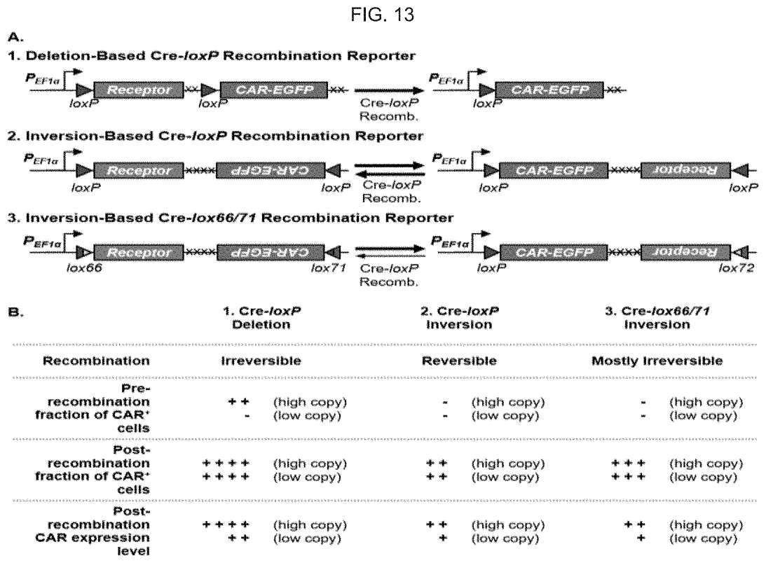

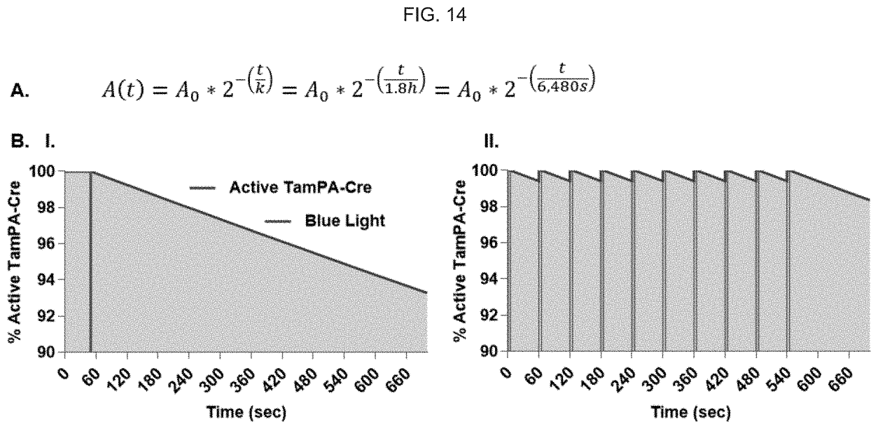

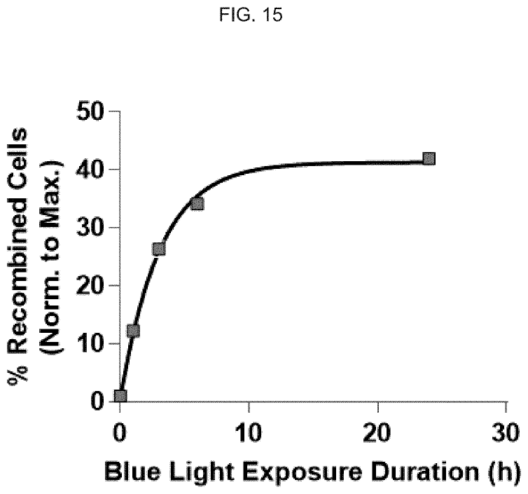

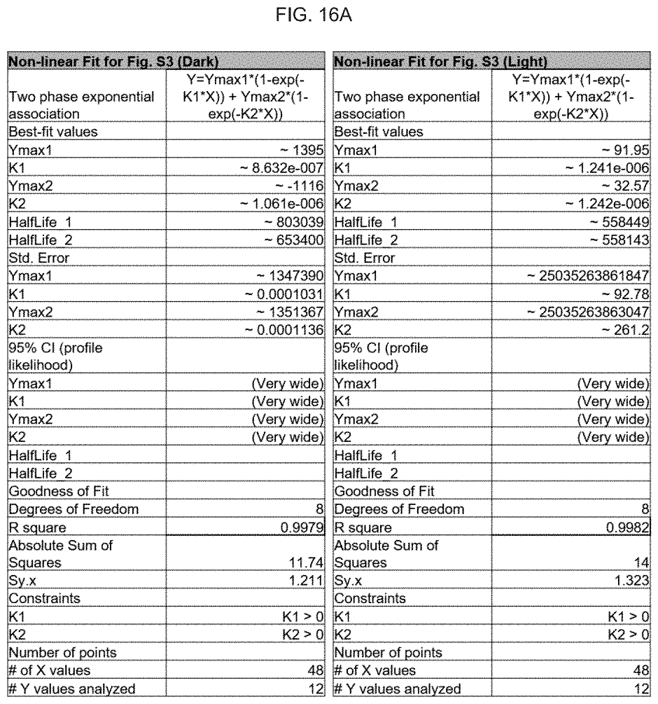

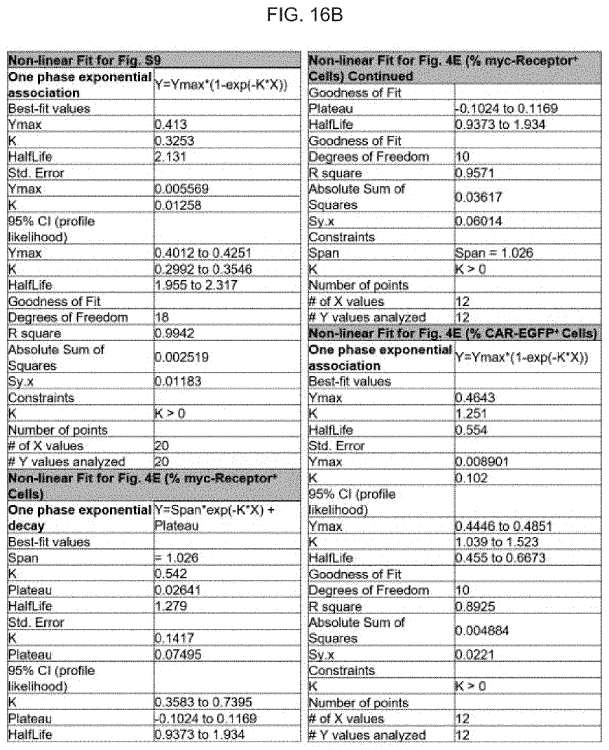

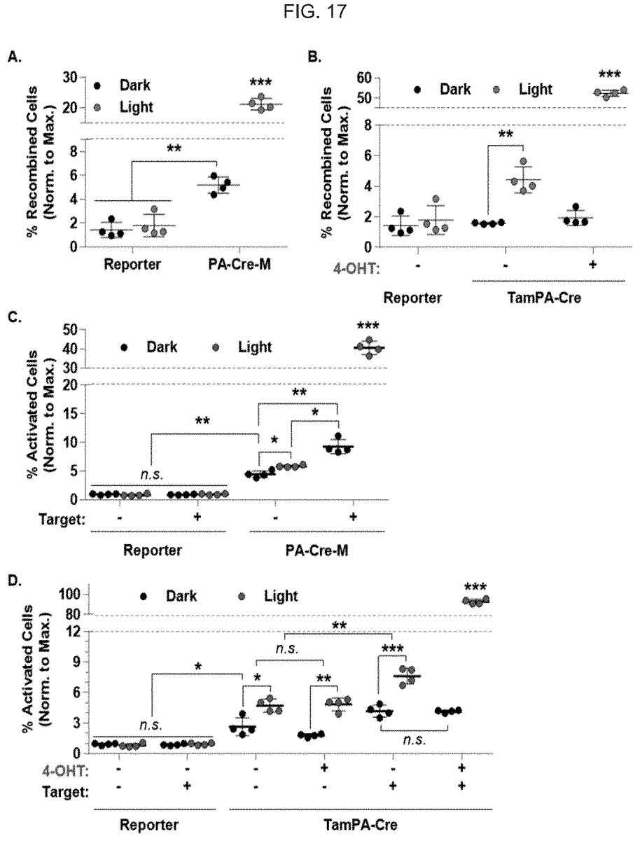

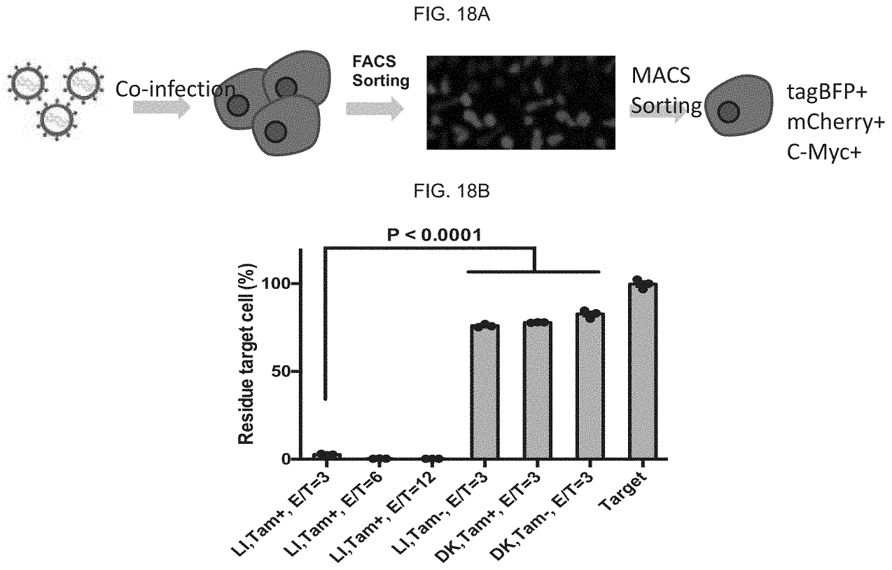

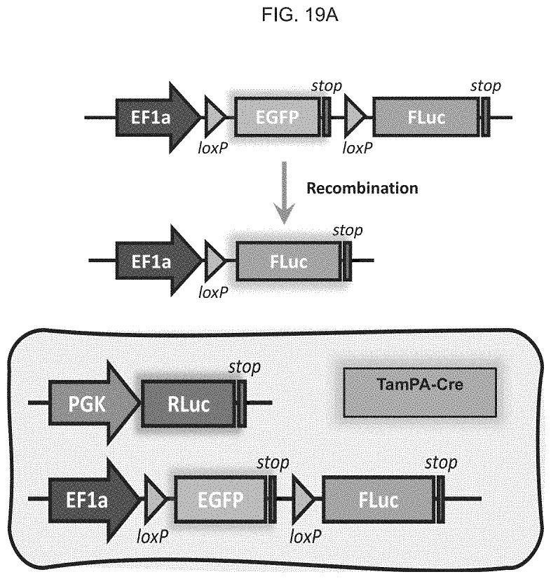

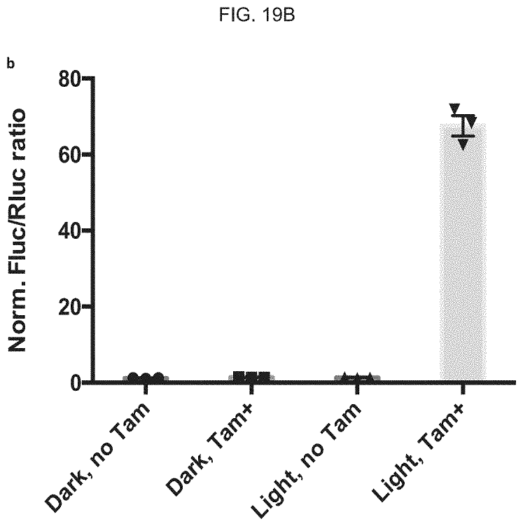

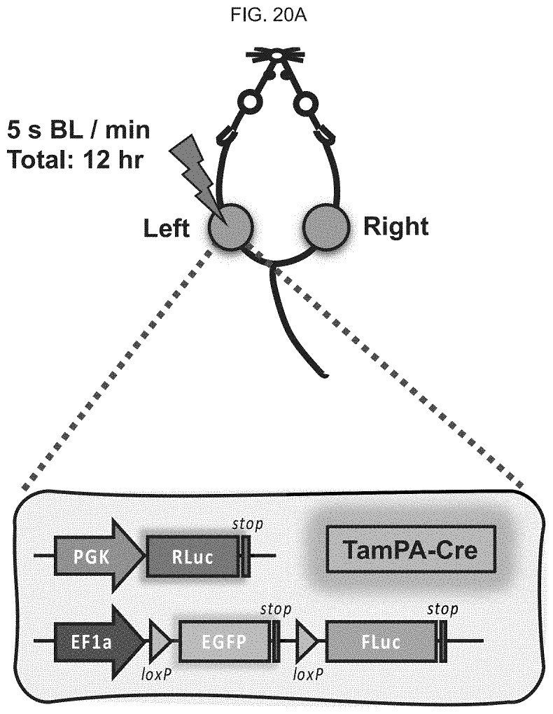

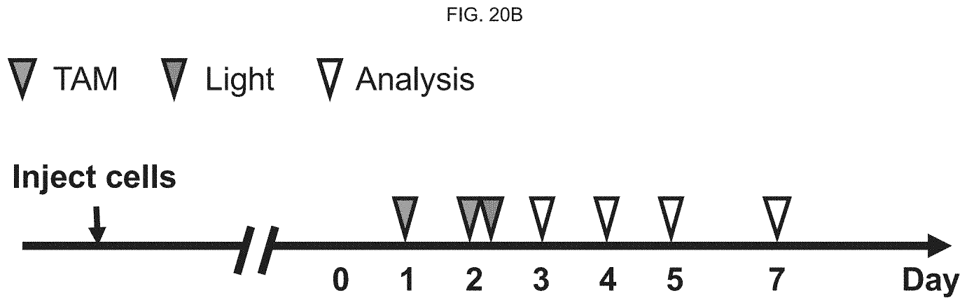

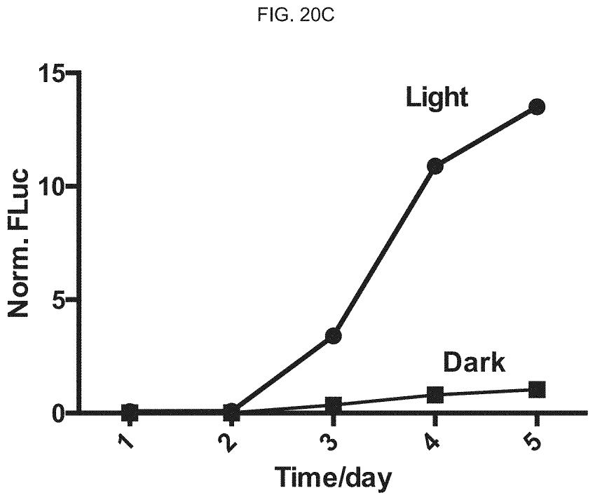

The left image schematically shows a person with Antigen1+ Antigen2+ cancerous (red) and healthy (purple) tissues in separate regions of the body, and engineered T cells express TamPA-Cre (ERT2-CreN-nMag and NLS-pMag-CreC) and the CAR Reporter genetic construct, consisting of a constitutive promoter driving expression of a foxed (purple) α-Antigen1 Receptor CDS with stop codons (black), followed by a-Antigen2 CAR (green), and upon intravenous introduction, the engineered T cells bind and localize to both cancerous (FIG. 1A) and healthy (FIG. 1D) Antigen1+ cells;

TamPA-Cre is inactive as its NLS-pMag-CreC and ERT2-CreN-nMag protein halves nuclear and cytosolically localized, respectively, after administration of tamoxifen, metabolite 4-hydroxytamoxifen (4-OHT) binds with ERT2-CreN-nMag to drive nuclear localization (FIG. 1B, FIG. 1E), priming TamPA-Cre;

Next, blue light is applied to the cancerous tissue region only (FIG. 1C) (healthy tissue shown in FIG. 1F), inducing nMag-pMag heterodimerization which restores active TamPA-Cre recombinase activity within the nucleus; the foxed α-Antigen1 Receptor CDS in the CAR Reporter is excised through Cre-loxP recombination along with its stop codons, thus allowing for α-Antigen2 CAR expression;

The T cell is finally activated upon CAR-mediated binding to Antigen2. T cells localized to the healthy tissue region (FIG. 1F) are not exposed to blue light and thus do not express α-Antigen2 CAR, effectively protecting healthy cells that express both Antigenl and Antigen2;

FIG. 1G illustrated a Boolean logic representation of an exemplary AND-gated TamPA-Cre system: (1) T cells first bind to cells expressing Antigen1 via the Receptor; (2) then, TamPA-Cre is be primed with tamoxifen before receiving localized (3) blue light stimulation in the cancerous tissue region, driving Cre-loxP recombination and CAR expression; (4) T cell activation is triggered when Antigen2 is recognized by the CAR; and

FIG. 1H is a detailed schematic of an exemplary CAR Reporter as provided herein.

FIG. 2A-E illustrate an evaluation of exemplary photoactivatable and tamoxifen-dependent Cre recombinase systems:

FIG. 2A is a schematic representation of the EGFP Reporter genetic construct before and after Cre-loxP recombination; the hEF1α promoter drives mCherry expression. During Cre-loxP recombination, the foxed mCherry is irreversibly excised along with its stop codons (XX), thus allowing for EGFP expression; the normalized percentage of recombined cells was calculated for EGFP Reporter HEK293T transiently transfected with the following constructs which did (Light) or did not (Ambient, Dark) receive the indicated blue light stimulation (473±29 nm), and FIG. 2B-D graphically illustrate: FIG. 2B shows PA-Cre-C, PA-Cre-M, or Cre PA constructs, (30 W/m², 30 s) (n=3), FIG. 2C shows PA-Cre-M or Cre constructs, (15 W/m², is per min, 24 h) (n=4), and FIG. 2D shows ERT2-Cre-ERT2 or Cre constructs, (15 W/m², is per min, 24 h) (n=4); reporter is the untransfected EGFP Reporter HEK293T cell line, blue light stimulation started and flow cytometry measurements taken 24 h and 72 h post-transfection, respectively, the percentage of recombined HEK293T cells (normalized to maximal recombination) is 100%*(% of EGFP⁺ cells)/(mean % of EGFP⁺ cells in corresponding Cre groups); and

FIG. 2E illustrates representative time-lapse fluorescence microscopy images of HEK293T cells transiently expressing ERT2-mCherry before and after the addition of nuclear-localizing 4-OHT (500 nM) (imaged every 2 min, 100× mag., scale bar=10 μm, n=6 independently measured cells).

FIG. 3A-E illustrate the design and optimization of an exemplary TamPA-Cre system in HEK293T cells:

FIG. 3A illustrates a schematic of TamPA-Cre as a single genetic construct with codon-diversified pMag and marker P2A-mCh (top), or as two genetic constructs: ERT2-CreN-nMag with marker P2A-mCh (bottom left) and NLS-pMag-CreC with marker tBFP-P2A (bottom right);

FIG. 3B is a schematic depicting an exemplary mechanism of the tamoxifen-AND blue-light-gated TamPA-Cre system;

FIG. 3C is a schematic illustrating two different tamoxifen and blue light stimulation protocols, each providing the same total amount of blue light energy (473±29 nm, not to scale); Protocol A: 3 hours (h) of continuous blue light stimulation (5 W/m²) started immediately after the addition of 4-OHT (500 nM), and Protocol B: 24 h of pulsatile blue light stimulation (5 W/m², 7.5 s per min) started 3 h after 4-OHT addition;

FIG. 3D graphically illustrates the percentage of recombined TamPA-Cre⁺ EGFP Reporter HEK293T cells (normalized to maximal recombination) which were (Light) or were not (Ambient, Dark) subjected to Protocol A or B (n=3) of FIG. 3C; and

FIG. 3E graphically illustrates the number of recombined cells after recombination in TamPA-Cre-nH1⁺ EGFP Reporter HEK293T cells subjected (Light) or not (Ambient, Dark) to Protocol B (n=3) of FIG. 3C; reporter is untransfected EGFP Reporter HEK293T cell line (n=3), blue light stimulation started and flow cytometry measurements taken 24 h and 72 h post-transfection, respectively; percentage of recombined HEK293T cells (normalized to maximal recombination)=100%*(% of EGFP⁺ cells)/(mean % of EGFP⁺ cells in corresponding Cre groups).

FIG. 4A-E illustrate the optimization and characterization of an exemplary TamPA-Cre system in Jurkat T cells:

FIG. 4A illustrates a schematic of an exemplary CAR Reporter construct before and after TamPA-Cre-mediated Cre-loxP recombination; the hEF1α promoter initially drives myc-α-CD38-Receptor expression; during Cre-loxP recombination, the foxed myc-α-CD38Receptor (with its stop codons, XX) is irreversibly excised allowing for α-CD19CAR-EGFP expression as illustrated by brightfield and EGFP (enhanced green fluorescent protein) images illustrated in FIG. 4B (Jurkat T cell, 100×, scale bar=10 μm);

FIG. 4C is a schematic illustrating tamoxifen (500 nM) and blue light stimulation (473±29 nm): Protocol E: (5 W/m², 5 s per min, 24 h) started 3 h after 4-OHT addition;

FIG. 4D graphically illustrates the percentage of recombined TamPA-Cre⁺ CAR Reporter Jurkat T cells (normalized to maximal recombination) exposed to Protocol E (FIG. 4C) over the course of 0, 1, 3, 6, or 24 h (n=4); and

FIG. 4E graphically illustrates a normalized percentage of myc-Receptor⁺ and CAR-EGFP⁺ TamPA-Cre⁺ CAR Reporter Jurkat T cells stimulated by Protocol E (FIG. 4C), measured 1, 3, and 5 days after start of blue light stimulation, fitted with exponential decay and association trendlines (GraphPad, Table S2) (n=4), reporter is CAR Reporter Jurkat T cell line. CAR-EGFP flow cytometry measurements taken 72 h after the start of blue light stimulation; percentage of recombined (% CAR-EGFP⁺) Jurkat T cells (normalized to maximal recombination) is 100%*(% of CAR-EGFP⁺ cells)/(initial % of CAR Reporter cells, measured via myc).

FIG. 5A-F illustrate how an exemplary TamPA-Cre system drives CAR-mediated T cell activation:

FIG. 5A is a schematic of the tamoxifen- and blue light-induced TamPA-Cre system in a CAR Reporter T cells driving recombination and CAR-mediated T cell activation upon binding to a TAA⁺ Target cells, the percentage of recombined cells (normalized to maximal recombination) is graphically illustrated in (FIG. 5B) PA-Cre-M⁺ and (FIG. 5C) TamPA-Cre⁺ CAR Reporter Jurkat T cells that did (+4-OHT) or did not (−4-OHT) receive tamoxifen (500 nM) stimulation, and did (Light) or did not (Dark) receive blue light stimulation (473±29 nm) as outlined in Protocol E (n=4); the percentage of activated cells (normalized to maximal recombination) of samples from (FIG. 5B) and (FIG. 5C) that were (+Target) or were not (−Target) co-incubated with CD19⁺ Target cells (1:1), as graphically illustrated in (FIG. 5D) and (FIG. 5E) respectively (n=4); and

FIG. 5F illustrates a heat map summary of T cell activation in Reporter, PA-Cre-M, and TamPA-Cre groups, with higher and lower efficiencies shown in red and green, respectively; reporter=CAR Reporter Jurkat T cell line; co-incubation started and flow cytometry measurements taken 48 h and 72 h after the start of blue light stimulation, respectively; percentage of recombined Jurkat T cells (normalized to maximal recombination) is 100%*(% of CAR-EGFP⁺ cells)/(initial % of CAR Reporter cells, measured via myc); percentage of activated Jurkat T cells (normalized to maximal recombination) is 100%*(% of CD69⁺ cells)/(initial % of CAR Reporter⁺ cells, measured via myc).

FIG. 6 illustrates a truth table highlighting potential on-target off-tumor toxicity risks for several engineered CAR T cell system. CAR T cell systems target localized cancerous cells that express Antigenl and/or Antigen2. Expected CAR-mediated T cell activation is given as an output for each unique combination of inputs, noting incidents of on-target on-tumor (blue) and on-target off-tumor toxicities (red). Blue light stimulation is only applied locally at the tumor site. T cells expressing the basic α-Antigen1 CAR will trigger on-target off-tumor toxicity against any Antigen1⁺ healthy cells along with cancer cells. The ON-Switch α-Antigen1 CAR system¹ relies on a diffusible small molecule to restore CAR function. Small molecules, however, are at risk of diffusing throughout the body, leaving Antigen1⁺ healthy cells susceptible to on-target off-tumor toxicity. The cytosolic costimulatory and activating domains of CAR can be split into 2 proteins, each with a unique antigen recognition domain. CAR function is restored upon binding to Antigen1⁺ Antigen2⁺ targeted cancer cells.² However, the split CAR will also trigger on-target off-tumor toxicity against any Antigen1⁺ Antigen2⁺ healthy cells. The synNotch receptor localizes T cells to all Antigen1⁺ cells, after which α-Antigen2 CAR is automatically expressed,³ similarly risking on-target off-tumor toxicity against Antigen1⁺ Antigen2⁺ healthy cells. The TamPA-Cre system localizes T cells to all Antigen1⁺ cells, but α-Antigen2 CAR is only expressed after global tamoxifen administration and localized blue light stimulation. As long as Antigen1⁺ Antigen2⁺ healthy cells are not colocalized with cancer cells, they are not at risk for on-target off-tumor toxicity.

FIG. 7 (referred to as FIG. S1 in Example 1, below) illustrates schematic representations of exemplary genetic constructs:

FIG. 7A schematically illustrates exemplary pMA002) PA-Cre-C:⁴pSin-mPGK-mCherry-P2A-CIBICreC(106-343)-IRES-CRY2(L348F)CreN(19-104);

FIG. 7B-C schematically illustrates exemplary: pMA005) PA-Cre-M⁵ in one vector: pSin-hEF1α-CreN(19-59)-NLS-P2A-NLS-dpMag-CreC(60-343) FIG. 7B, or in two vectors: (C1, pMA003) pSin-hEF1α-CreN(19-59)-nMag-NLS-P2A-mCherry-IRES-PuroR and (C2, pMA004)pSin-hEF1α-tBFP-P2A-NLS-pMag-CreC(60-343)-IRES-PuroR FIG. 7C;

FIG. 7D schematically illustrates exemplary: pMA001) Cre: pSin-hEF1α-Cre-2xP2A-mCherry; (E, pMA006) ERT2-Cre-ERT2: pSin-hEF1α-ERT2-Cre-ERT2;

FIG. 7F schematically illustrates: pMA007) ERT2-mCh: pSin-hEF1α-ERT2-mCherry;

FIG. 7G-H schematically illustrate exemplary: pMA013) TamPA-Cre in one vector: pSin-hEF1α-ERT2-CreN(2-59)-nMag-P 2A-NLS-dpMag-CreC(60-343)-P 2A-mCherry; or (FIG. 7H) in two vectors: (H1, pMA011)pSin-hEF1α-ERT2-CreN(2-59)-nMag-P2A-mCherry-IRES-PuroR and (C2, pMA004);

FIG. 71 schematically illustrates exemplary: TamPA-Cre-nH1 in two vectors: (II, pMA012)pSin-hEF1α-ERT2-CreN(2-59)-nMagHigh1-IRES-PuroR and (C2, pMA004);

FIG. 7J schematically illustrates exemplary: pMA014) EGFP Reporter: pSin-hEF1α-loxp-mCherry-2stop-loxP-EGFP-IRES-PuroR; and

FIG. 7K schematically illustrates exemplary: pMA022) CAR Reporter pSin-hEF1α-loxp-myc-α-CD38-Receptor-2stop-loxP-α-CD19-CAR-EGFP-IRES-PuroR. dpMag=codon diversified pMag.

FIG. 8A-C (referred to as Figure S2 in Example 1) illustrates an exemplary design, and characterization of an exemplary blue light stimulation apparatus:

FIG. 8A illustrates images of exemplary apparatus used to practice methods as provided herein, for example, to distribute light from a single LED evenly across a standard 24-well plate of cells, we built the blue light stimulation apparatus with an LED placed at a defined distance from a polypropylene light-diffusion film: (I) Close up of the mounted blue LED covered in a protective layer of transparent epoxy; (II) Dual Lock Reclosable Fasteners (3M, yellow) were adhered to the back of the LED (not shown) and in the center bottom of a deep, polyester white-walled container with a clear PET plastic lid and synthetic rubber gasket; small plastic Decorating Clips (Command, white) were used to hold the LED wire along the inner walls of the container, out of the light path, a small notch was cut into the white container to minimize strain on the wire when the lid was fastened; (III) A polypropylene static-cling frosted window film was cut to size and placed on the inside of the lid to work as a light-diffusion film; (IV) The LED was mounted in the bottom center of the container and the wires secured with clips, in the background, the wire connects to the intensity-adjustable blue LED control system (Phillip Kyriakakis); (V) the lid was fastened onto the blue light box and the blue light is turned on to demonstrate function, the cell culture place was centered inside a lidless black plastic box, which was fitted to the beveled lid of the blue light box; (VI) After blue light intensity was measured and adjusted using a power meter, the blue light box was flipped upside down and centered on top of the cell culture plate in the lidless black plastic box;

During experiments, this FIG. 8A exemplary configuration is assembled inside of a humidified 37° C., 5% CO₂ cell culture incubator; finally, blue light exposure times and patterns are entered into the LED control system's LabVIEW-based software outside of the incubator.

FIG. 8B illustrates spectral evaluation of the blue LED used to stimulate photoactivatable Cre-loxP recombination; the LED was measured by mounting it onto a cell culture plate, then using a microplate reader (Infinite M1000 Pro, Tecan) to measure the intensity of the LED at different wavelengths (1 nm intervals); peak intensity wavelength occurred at 473 nm (bandwidth=29 nm); and

FIG. 8C graphically illustrates the characterization of light distribution showing more even distribution with greater distance from the LED light and with scattering from the diffusion film lid: the blue light box was set up shown in A-VI, such that light intensity could be measured at the approximate location experience by cells in each well of a 24-well plate; the shortest distance between the light source and center of the 24-well plate cell culture plane was approximately 15.5 cm; the lid with the diffusion filter was located a minimum of 10.1 cm from the light source; other exemplary designs can include an array of multiple LEDs to further improve light distribution.

FIG. 9A-B (referred to as Figure S3 in Example 1) illustrates additional characterization of an exemplary PA-Cre-M system:

FIG. 9A graphically illustrates excessive expression of PA-Cre-M drives high levels of spontaneous recombination; CAR Reporter Jurkat T cells were transiently transfected via electroporation with PA-Cre-M; 24-hours post-transfection, cells were (Light) or were not (Dark) subjected to pulsatile blue light stimulation for 24 h (50 W/m², 1 s per min); trend lines fitted to Dark (black) and Light (green) groups were created using a two-phase exponential association model; at low PA-Cre-M expression levels (as measured by the intensity of the mCherry marker), the difference between the Light and Dark groups is small; with increasing expression level, light-induced PA-Cre-M drives increasing levels of recombination while cells kept in the dark remain relatively low; however, at higher expression levels, the difference between Recombination in Dark and Light groups begins to narrow as cells in the Dark groups become more susceptible to spontaneous background recombination; data is from the analysis of one experiment, but represents a common trend seen across several different experiments in which PA-Cre-M was variably expressed; and

FIG. 9B graphically illustrates Cre-loxP recombination cannot be driven by CreN-nMag-NLS or NLS-pMag-CreC alone. EGFP Reporter HEK293T cells transiently transfected with CreN-nMag-NLS-P2A-mCherry (CreN-nMag-NLS), or tBFP-P2A-NLS-pMag-CreC (NLS-pMag-CreC) constructs either were (Light) or were not (Dark, Ambient) exposed to blue light stimulation (5 W/m², 7.5 s per min, 24 h) (n=5), across Reporter, CreN-nMag-NLS, and NLS-pMag-CreC groups, there is no significant difference seen in any light condition (Dark, Ambient, Light), Blue light stimulation started and flow cytometry measurements taken 24 h and 72 h post-transfection, respectively; Percentage of recombined HEK293T cells (normalized to maximal recombination)=100%*(% of EGFP⁺ cells)/(mean % of EGFP⁺ cells in corresponding Cre groups).

FIG. 10 (referred to as Figure S4 in Example 1) illustrates diversification of the pMag CDS:

FIG. 10A illustrates DNA sequence alignment of nMag and pMag (Serial Cloner 2.6.1) from PA-Cre-M⁵ showing that the CDS for each are nearly identical and are thus susceptible potential recombination introduced by lentiviral gene transfer⁶, and,

FIG. 10B illustrates DNA sequence alignment of pMag with codon-diversified dpMag (Serial Cloner 2.6.1); reduced sequence similarity helps prevent potential recombination introduced by lentiviral gene transfer⁶, Codon diversification of the pMag CDS was done by hand and synthesized dpMag dsDNA (Integrated DNA Technologies) was used for subsequent molecular cloning; corresponding Cre groups.

FIG. 11A-C (referred to as Figure S5 in Example 1) illustrates optimizing TamPA-Cre recombination efficiency through different tamoxifen and blue light stimulation protocols:

FIG. 11A-B right images are schematics illustrating the tamoxifen and blue light stimulation protocols and the normalized percentage of recombined EGFP Reporter HEK293T cells transiently transfected with TamPA-Cre or Cre constructs that were (Light) or were not (Dark) exposed to blue light stimulation outlined in (FIG. 11A) Protocol C: (5 W/m², 3 h) started 8 h after 4-OHT addition (n=4), or (FIG. 11B) Protocol D: (15 W/m², 7.5 s per min, 24 h) started with 4-OHT addition; delaying blue light stimulation after 4-OHT addition and changing to a pulsatile blue light pattern improved TamPA-Cre performance, and resulting data are graphically illustrated in the images to the right of schematic FIG. 11A-B;

FIG. 11C graphically illustrate, for comparison, EGFP Reporter HEK293T cells transiently transfected with PA-Cre-M or Cre constructs were (Light) or were not (Dark, Ambient) exposed to Protocol B: (5 W/m², 7.5 s per min, 24 h) started 3 h after 4-OHT addition (n=3), Reporter=untransfected EGFP Reporter HEK293T cell line, Blue light stimulation started and flow cytometry measurements taken 24 h and 72 h post-transfection, respectively; Percentage of recombined HEK293T cells (normalized to maximal recombination)=100%*(% of EGFP⁺ cells)/(mean % of EGFP⁺ cells in corresponding Cre groups).

FIG. 12A-F (referred to as Figure S6 in Example 1) illustrates CAR-mediated T cell activation is antigen specific at low CAR expression levels:

FIG. 12A is a schematic representation of the α-CD 19CAR-EGFP (pMA017) and the myc-α-CD38Receptor-EGFP (pMA020) genetic constructs; the hEF1α promoter drives constitutive expression of α-CD 19CAR or myc-α-CD38Receptor (pSin-hEF1α-α-CD19-CAR-ggsggt-EGFP-IRES-PuroR, or pSin-hEF1α-myc-α-CD38Receptor-ggsggt-EGFP-IRES-PuroR, respectively);

FIG. 12B graphically illustrates Jurkat T cells and α-CD19-CAR-EGFP⁺ Jurkat T cells were co-incubated with an equal number of either CD19⁻ K562 Target cells, CD19⁺ Toledo Target cells, or no Target cells for 24 h after which the normalized percentage of activated (% CD69⁺) Jurkat T cells was measured via flow cytometry (n=3); oOnly α-CD19CAR⁺ Jurkat T cells co-incubated with CD19⁺ Toledo Target cells were significantly activated (81.1±0.35%); co-incubation with either no Target cells or CD19⁻ K562 Target cells yielded similar results, indicating that CAR-mediated T cell activation is not affected by the presence or absence of TAA⁻ Target cells;

FIG. 12C graphically illustrates representative histograms from flow cytometry showing that a greater portion of Jurkat T cells expressing high levels of α-CD19-CAR-EGFP undergo non-specific T cell activation in culture (CD69⁺, green) compared to those expressing low levels of α-CD19-CAR-EGFP (blue);

FIG. 12D graphically illustrates representative histograms from flow cytometry showing that α-CD19-CAR-EGFP⁺ Jurkat T cells both (I) bind to and (II) are activated by CD19⁺ Toledo Target cells, while myc-α-CD38-Receptor⁺ Jurkat T cells (III) bind to but (IV) are not activated by CD38⁺ Toledo Target cells;

FIG. 12E graphically illustrates representative histograms from flow cytometry measurement of CAR Reporter Jurkat cell line (red) transduced with either CreN-nMag-NLS-P2A-mCh (yellow), ERT2-CreN-nMag-P2A-mCh (green), or tBFP-P2A-NLS-pMag-CreC (blue) constructs; all CAR Reporter Jurkat T cells express myc-α-CD38Receptor (left) and do not express α-CD19-CAR-EGFP (right). Non-transduced Jurkat T cells (black) are shown for reference; and

FIG. 12F graphically illustrates data where PA-Cre-M⁺ and TamPA-Cre⁺ CAR Reporter Jurkat cell lines were exposed to the Ambient light condition; the percentage of recombined cells (% CAR-EGFP⁺) was measured before and after via flow cytometry (n=3); after 48 h, PA-Cre-M had driven a significant increase in recombination while no such change was seen in TamPA-Cre groups; this increase in PA-Cre-M groups is likely due to a combination of spontaneous and ambient light-driven recombination; Reporter =CAR Reporter Jurkat T cell line; Recombination=% CAR-EGFP⁺ cells, and Activation=% CD69⁺ cells, where 100% represents maximal recombination efficiency; Percentage of recombined Jurkat T cells (normalized to maximal recombination)=100%*(% of CAR-EGFP⁺ cells)/(initial % of CAR Reporter⁺ cells, measured via myc).

FIG. 13A-B (referred to as Figure S7 in Example 1) illustrates testing of different exemplary Cre-loxP CAR Reporter designs:

FIG. 13A schematically illustrates three different exemplary Cre-loxP CAR Reporter constructs that were evaluated: (1) deletion-based (irreversible), (2) inversion-based (reversible), and (3) inversion based with loxP mutant sites 1ox66 and lox71 (mostly irreversible); and FIG. 13B illustrates a qualitative summary of Cre-, PA-Cre-, and TamPA-Cre-mediated recombination experiments that were conducted in both HEK293T and Jurkat T cells expressing high (high copy) or low (low copy) levels of each CAR Reporter construct; while inversion-based CAR Reporters had the advantage of preventing all background CAR-EGFP expression before recombination, the deletion-based CAR Reporter functioned similarly at low copy number while providing more robust expression levels of CAR-EGFP after recombination; in the ideal CAR Reporter design, the receptor would continue to be expressed after recombination, however, due to lentiviral packaging size constraints and a desire to minimize the number of individually introduced components, in one embodiment only one promoter is used, and as efficiency improves, other gene insertion methods (for example CRISPR-Cas9) also can be used allow for larger, more complex designs.

FIG. 14A-B (also referred to as Figure S8 in Example 1) illustrates pulsatile blue light stimulation protocols improve Cre-loxP recombination efficiency due to slow nMag-pMag dissociation kinetics:

FIG. 14A illustrates a mathematical model showing how blue light-induced heterodimerization between nMag and pMag proteins occurs rapidly, but nMag-pMag heterodimers dissociate exponentially with a half-life of approximately 1.8 h upon the removal of light⁷, from an initial heterodimer concentration of A₀, the remaining heterodimer concentration after the blue light stimulus is removed can be calculated as a function of time using the exponential decay equation, where the half-life k is equal to 1.8 h (6,480 s); such slow degradation kinetics maintain the percentage of active TamPA-Cre above 99.0% up to 93 s after blue light is removed; and

FIG. 14B graphically illustrates plots that track the theoretical percentage of active TamPA-Cre in tamoxifen-induced cells given (left image I) 50 s of continuous blue light stimulation or given (right image II) ten 5-second pulses of (equivalent intensity) blue light stimulation, each pulse separated by 55 s of darkness (a shortened version of Protocol E, FIG. 4C); 700 s after the beginning of each blue light stimulation protocol, only 93.28% of maximal active TamPA-Cre levels remain given 10 s of continuous light, while 98.36% of maximal active TamPA-Cre is still present to drive recombination given the pulsatile protocol; the area under the % Active TamPA-Cre curve (shaded red) is proportional to the probability that a given cell will undergo Cre-loxP recombination over a given time interval; from these graphs, it is obvious that the area under to curve for the pulsatile blue light stimulation (II) is greater than for continuous blue light stimulation (I), thus increasing the chances that these cells will undergo TamPA-Cre-mediated Cre-loxP recombination.

FIG. 15 (also referred to as Figure S9, in Example 1) illustrates Cre-loxP recombination in TamPA-Cre⁺ CAR Reporter Jurkat T cells plateaus between 6 h and 24 h of blue light stimulation; alternate graphical representation of FIG. 4D with non-linear fit exponential growth trendline; percentage of recombined Jurkat T cells (normalized to maximal recombination)=100%*(% of CAR-EGFP⁺ cells)/(initial % of CAR Reporter cells, measured via myc).

FIG. 16A-B, illustrating Table S2, shows Non-linear Fit Trendline Information; parameters of non-linear fit trendlines in Fig. S3, S9, and 4E (GRAPHPAD PRISM 7.04™).

FIG. 17A-D (also referred to as Figure S10 in Example 1) illustrates data showing expanded lower range of plots in FIG. 5B-E; FIG. 17A=FIG. 5B, FIG. 17B=FIG. 5C, FIG. 17C=FIG. 5D, FIG. 17=FIG. 5E; percentage of recombined Jurkat T cells (normalized to maximal recombination)=100%*(% of CAR-EGFP⁺ cells)/(initial % of CAR Reporter cells, measured via myc); percentage of activated Jurkat T cells (normalized to maximal recombination)=100%*(% of CD69⁺ cells)/(initial % of CAR Reporter cells, measured via myc).

FIG. 18A-B illustrate in vitro killing assays for primary human T cells engineered with an exemplary TamPA-Cre system as provided herein:

FIG. 18A schematically illustrates a protocol where primary human T cells were infected with lentiviral vectors to express an exemplary Reporter genetic cassette, for example, the exemplary TamPA-Cre (ERT2-CreN-nMag and NLS-pMag-CreC) and the CD19CAR Reporter genetic cassette; engineered cells were sorted based on tagBFP and mCherry fluorescence (FACS) and c-Myc tag staining (MACS); and

FIG. 18B graphically illustrates data where the sorted engineered cells were treated with or without 4-OHT (Tam+ or Tam−) and with or without blue light stimulation (LI, or DK: Dark); the treated cells were then co-cultured with CD19+ target Nalm-6 cells (engineered to express firefly luciferase constitutively) for 24 hr at different effector/target (E/T) ratios as shown; the percentage of residue target cell after co-culture in each group was normalized to target-cell only group (Target, defined as 100%) based on firefly luciferase measurement; the reduction fold change of target cell between DK,Tam− and LI,Tam+ was shown in red. Error bars, standard deviation (n=3). Two-tailed Student's T test.

FIG. 19A-B illustrate construction and characterization of exemplary reporter cell line for in vivo testing of TamPA-Cre:

FIG. 19A upper image schematically illustrates that Jurkat cells were infected with lentiviral vectors to express TamPA-Cre (ERT2-CreN-nMag and NLS-pMag-CreC), Renilla luciferase, and the firefly luciferase reporter (loxP-EGFP-stop-loxP-Fluc); and, the lower image schematically illustrates TamPA-Cre mediated recombination; before recombination, the reporter cassette only expresses EGFP but not firefly luciferase, after TamPA-Cre mediated loxP recombination, EGFP will be removed from the reporter cassette, and firefly luciferase will be expressed; and

FIG. 19B graphically illustrates data showing the in vitro characterization of the exemplary reporter cell line; engineered Jurkat reporter cells were treated with or without 4-OHT (Tam+ or no Tam) and with or without blue light stimulation (Light or Dark). Error bars, standard deviation (n=3).

FIG. 20A-C illustrate an exemplary model of tamoxifen-gated light-inducible gene activation in vivo:

FIG. 20A schematically illustrates a mouse model for testing TamPA-Cre system for gene activation; a total of 5 million engineered Jurkat reporter cells were subcutaneously injected on both left and right sides of an NSG mouse; to demonstrate the effect of light on gene activation, only the left side will be illuminated (5 s per min, for 12 hr);

FIG. 20A schematically illustrates an exemplary experiment timeline: the mouse was injected with 100μ.1 tamoxifen/corn oil solution (20 mg/mL) on day 1 and day 2 as shown, light stimulation was performed on day 2 after tamoxifen injection, bioluminescence from firefly luciferase and Renilla luciferase was monitored; and

FIG. 20C graphically illustrates the light-induced gene expression of firefly luciferase: Normal Fluc is shown as a function of time/day: Firefly luciferase activity was normalized against Renilla luciferase; values from illuminated and non-illuminated sides are indicated.

Like reference symbols in the various drawings indicate like elements.

### DETAILED DESCRIPTION

In alternative embodiments, provided are compositions, including products of manufacture and kits, and methods, for remotely-controlled and non-invasive manipulation of physiologic or genetic processes and/or protein expression in live cells in vivo, for example, immune cells such as T cells, monocytes/macrophages, dendritic cells, natural killercells, for example, for the controlled expression of recombinant nucleic acids or proteins such as for example, chimeric T cell or NK cell receptors, chimeric immunoreceptors or chimeric antigen receptors (CARs), for the manipulation of physiologic processes in the cell or for the correction of pathological processes (for example, non-specific targeting of the CAR-T cells against normal/nonmalignant tissues) and/or for control of therapeutic outcomes, for example, engineered immune cells (for example, T cells or NK cells) expressing CARs targeting specific cancers cells and killing them.

In alternative embodiments, provided are compositions and methods for the manipulation or correction of pathological processes, for example, for eradicating tumors and cancers in human subjects, without limitation in penetration depth of an inducible signal, that comprise use of ultrasound stimulation. In alternative embodiment, provided are compositions and methods for inducing expression of nucleic acids, for example, genes, in immune cells such as T cells, monocytes/macrophages, dendritic cells, natural killercells and the like. In alternative embodiment, provided are compositions and methods for stimulating or inhibiting ligand-receptor interactions, including any surface molecular interaction, including but not limiting to inhibitory CTLA-4 and apoptotic Fas.

In alternative embodiments, provided are compositions and methods for the treatment, amelioration, prevention or eradication of a pathologic process or a pathology, a disease, an abnormal tissue, or an infection, for example, bacterial or viral infections, with a specific cell surface marker. In alternative embodiment, provided are compositions and methods for the controlled production of RNAs (including microRNA, long non-coding RNAs), and for the epigenetic and genetic modulation of molecules for the treatment, amelioration, prevention or eradication of a pathologic process, a disease, an abnormal tissue, or an infection.

In alternative embodiments, provided are engineered cells, for example, human cells, for example, immune cells, for example, T cells, capable of inducibly expressing a nucleic acid, for example, a protein encoding nucleic acid, for example, expressing a recombinant protein such as a chimeric antigen T cell receptor (CAR), by operatively linking a gene of interest, i.e., a gene (for example, a gene expressing a CAR), to a duel tamoxifen/blue light induced nucleic acid expression system.

Engineered chimeric antigen receptor (CAR) T cells as provided herein can detect and eradicate cancer cells within patients, and provides truly cancer-specific CAR-targeting cell surface antigens to prevent potentially fatal on-target off-tumor toxicity against other healthy tissues within the body. Engineered cells as provided herein can accomplish this by use of a novel tamoxifen-gated photoactivatable split-Cre recombinase optogenetic system, called TamPA-Cre, that features high spatiotemporal control to limit CAR T cell activity to the tumor site for immunotherapy applications. We created and optimized a novel dual switch, i.e., a genetic and gate switch, by integrating the features of tamoxifen-dependent nuclear localization (with the ERT2 domain) and blue-light-inducible heterodimerization of Magnet protein domains (nMag, pMag) into split Cre recombinase. Upon blue light stimulation following tamoxifen treatment, the TamPA-Cre system exhibits sensitivity to low intensity, short durations of blue light exposure to induce robust Cre-loxP recombination efficiency.

We demonstrate that this TamPA-Cre system can be applied to specifically control localized CAR expression and subsequently T cell activation. As such, we posit that CAR T cell activity can be confined to a solid tumor site by applying an external stimulus, with high precision of control in both space and time, such as light. Furthermore, the highly controllable TamPA-Cre system as provided herein can replace virtually any Cre-loxP system. Exemplary TamPA-Cre systems as provided herein are useful alternatives to CRE-ERT2 systems, for example, in mouse lines, where spontaneous Cre-loxP background recombination in vivo is already an established problem.

TamPA-Cre system as provided herein also are readily applicable for clinical applications, for example locally metastatic head/neck cancer, melanoma, or other skin cancers, typically at superficial location, as lights (for example, blue lights) can reach these locations after the local injection of the engineered controllable CAR T cells.

While current systems that induce CAR activity via the administration or release of small molecules or proteins have allowed for some degree of temporal control, their inability to localize CAR activation to the site of the solid tumor still allows for potential on-target off-tumor toxicity (see Supplementary Table 1). Light-induced optogenetic systems offer precise control over the dosage, duration, and location of stimulation—ideal for controlling CAR expression in both space and time. However, current optogenetic systems are often weakly regulated and allow for the premature CAR expression responsible for on-target off-tumor toxicity. The TamPA-Cre, a small molecule- and light-inducible split Cre recombinase optogenetic systems as provided herein address this issue by tightly regulating CAR expression. By fusing the cytosol-localizing mutant estrogen receptor ligand binding domain (ERT2) to the N-terminal half of split Cre(2-59aa)-nMag, the TamPA-Cre protein ERT2-CreN-nMag is physically separated from its nuclear-localized binding partner, NLS-pMag-CreC(60-343aa). Without tamoxifen to drive nuclear localization of ERT2-CreN-nMag, the typically high background of the photoactivation system lacking ERT2 is significantly suppressed.

In summary, we have developed a novel logic-gated optogenetic split Cre system by integrating both ERT2-fusion proteins with the blue light-inducible nMag-pMag heterodimerizing domains to drive robust Cre-loxP recombination with significantly suppressed background. Only after treatment with tamoxifen is the TamPA-Cre system primed to be activated by short pulses of low intensity blue light stimulation. The tamoxifen gate helps prevent the spontaneous Cre-loxP recombination within cells prior to specific blue light stimulation—a weakness of other photoactivatable Cre-loxP systems. Applying the TamPA-Cre system to our foxed CAR-Reporter construct in Jurkat T cells, we were able to precisely induce CAR expression and antigen-specific T cell activation. With its unique high spatiotemporal control over T cell activation, the TamPA-Cre system could be used to locally induce T cell effector functions against cancer cells in vivo while avoiding on-target off-tumor toxicity in TAA⁺ healthy tissues.

Exemplary TamPA-Cre systems as provided herein also offer improved spatiotemporal control over other engineered CAR systems, like SynNotchn and SUPRA CAR.⁴² Suicide switches⁴³⁻⁴⁴ and iCARs⁴⁵ can be further integrated into the robust photoactivatable systems provided herein to prevent potential on-target off-tumor toxicity caused by TamPA-Cre-activated CAR T cells leaving the stimulated region following tumor eradication. CRISPR-Cas9 technology can also help integrate large TamPA-Cre and engineered CAR T cell system designs into safe and effective loci in the genome.⁴⁶ Furthermore, while the highly controllable TamPA-Cre system can replace virtually any Cre-loxP system, we foresee that it will serve as a particularly useful alternative to CRE-ERT2 systems in mouse lines where spontaneous Cre-loxP background recombination in vivo is already an established problem.⁴⁷

Exemplary TamPA-Cre systems as provided herein are applicable for clinical applications, for example locally metastatic head/neck cancer, melanoma, or other skin cancers, typically at superficial location, as lights can reach these locations after the local injection of the engineered controllable CAR T cells.

In immunotherapy, the expression of engineered CAR on the cell surface enables T cells to recognize specific antigens on the target cell. This triggers T cell activation can eventually lead to the elimination of target cells. Clinical trials involving anti-CD19 CAR T cells against B-cell malignancies have shown promising results, demonstrating the therapeutic effects of CAR T cells in cancer treatment. In alternative embodiments, compositions and methods as provided herein address the problem that occurs upon perfusion of constitutively activated CAR T cells into patients, which may have lethal consequences due to the induced cytokine storm and ‘on-target, off tumor’ toxicity, by controlling the timing and location of the activation of the perfused CAR T cells.

In alternative embodiments, methods as provided herein can remotely and non-invasively activate any cell in vivo, including immune cells such as T cells, for example, CAR T cells, with precise spatial and temporal control. In alternative embodiments, methods as provided herein are used to remotely control other stimulatory or inhibitory ligand-receptor interactions, as well as any surface molecular interaction, including but not limiting to inhibitory PD-1, CTLA-4 and apoptotic Fas.

In alternative embodiments, methods as provided herein are used in the eradication of other diseases or abnormal tissues with specific surface markers. In alternative embodiments, methods as provided herein are used to treat or eradicate bacterial or viral infections.

In alternative embodiments, methods as provided herein are used are used for the controlled production of RNAs (including microRNA, long non-coding RNAs, antisense or miRNAs), for example, for the epigenetic and genetic modulation molecules for the treatment of diseases.

After administering or contacting the cell with tamoxifen, it is the tamoxifen metabolite 4-hydroxytamoxifen (4-OHT) that binds with the ERT2-CreN-nMag cytosolically localized protein to drive its nuclear localization to prime TamPA-Cre. Plasma 4-OHT levels are expected to decay over time, varying naturally with metabolic and clearance efficiency between different individuals. The plasma concentrations of tamoxifen declines with a terminal elimination half-life of about 5 to 7 hours, see for example, Fun B J, Jordan V C (1984) The pharmacology and clinical uses of tamoxifen. Pharmacol Ther 25(2): 127-205. Thus, 4-OHT levels will not remain at therapeutic levels over the course of days.

In alternative embodiments, blue light treated areas, particularly areas that may be triggered inadvertently by sunlight or artificial light, such as exposed skin, are covered locally from light until, for example, plasma tamoxifen levels test below the therapeutic threshold, or a calculated amount of time needed for metabolite 4-OHT plasma levels to fall below effective levels.

### CRISPR Systems for Genome Integration

In alternative embodiments, a CRISPR method is one alternate method of engineering the target cells (for example, for inserting the TamPA-Cre system in target cells), for example, as an alternative to using the exemplary lentivirus infection method also described herein.

For example, in alternative embodiments, a SpCas9 or equivalent protein and a guide RNA are expressed or delivered into a target cell, for example, through electroporation or equivalents. The CAR reporter as provided herein is included in a template plasmid which contains nucleic acid sequence homologous to the targeted insertion region of target cell genome, and is delivered into target cells, for example, through electroporation or equivalents. After CRISPR-mediated homology-directed repair, the CAR reporter is inserted in the target cell genome. Successful integration of the CAR reporter into the target cell genome can be verified by genotyping methods and/or function-based assays.

Regardless of which exemplary method is used (for example, lentivirus or CRISPR), the system is still a tamoxifen-gated photoactivatable split-Cre recombinase (TamPA-Cre) optogenetic system, meaning that in both cases Cre-lox will be utilized to express CAR protein from the CAR reporter nucleic acid sequence when the TamPA-Cre system is activated.

In alternative embodiments, for using CRISPR (for example, instead of the exemplary lentivirus) to engineer target cells, the components needed are: (1) SpCas9 protein (either protein form or nucleic acid form which can express SpCas9 protein when in cells); (2) a single guide RNA (sgRNA, either RNA form or nucleic acid form which can be transcribed into RNA when in cells); and (3) a template plasmid which contains the CAR reporter (including the exemplary Cre-loxP structure), flanked by nucleic acid sequence homologous to the targeted insertion locus/loci (determined by sgRNA) of genome of the cell. With all these components, the cells can go through CRISPR-mediated homology-directed repair, which can insert the CAR reporter in the genome.

In alternative embodiments, any CRISPR system can be used to practice embodiments as provided herein, for example, as described in U.S. Pat. Nos. 10,767,168; 10,760,081; 10,745,716; 10,711,285; 10,711,284; 10,668,173; and/or 10,577,630.

Products of manufacture and Kits

Provided are products of manufacture and kits for practicing methods as provided herein; and optionally, products of manufacture and kits can further comprise instructions for practicing methods as provided herein.

Any of the above aspects and embodiments can be combined with any other aspect or embodiment as disclosed here in the Summary and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 90%, 95%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

Unless stated otherwise in the Examples, all recombinant DNA techniques are carried out according to standard protocols, for example, as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Other references for standard molecular biology techniques include Sambrook and Russell (2001) Molecular Cloning: A Laboratory Manual, Third Edition, Cold Spring Harbor Laboratory Press, NY, Volumes I and II of Brown (1998) Molecular Biology LabFax, Second Edition, Academic Press (UK). Standard materials and methods for polymerase chain reactions can be found in Dieffenbach and Dveksler (1995) PCR Primer: A Laboratory Manual, Cold Spring Harbor Laboratory Press, and in McPherson at al. (2000) PCR—Basics: From Background to Bench, First Edition, Springer Verlag, Germany.

### Example 1: Acoustic Thermogenetics for Remote-Controlled Gene Expression and T Cell Activation

This example demonstrates that methods and recombinantly engineered cells as provided are effective and can be used to treat cancer and tumors.

An exemplary mechanism of an exemplary TamPA-Cre activation system is illustrated in FIG. 1 and FIG. 3B. Without tamoxifen- or blue light stimulation, TamPA-Cre is inactive with ERT2-CreN-nMag and NLS-pMag-CreC proteins spatially segregated to the cytosol and nucleus, respectively. This physical separation prevents the spontaneous, concentration-dependent nMag-pMag dimerization seen in the PA-Cre-M system (which lacks ERT2) and thus suppresses the unwanted background levels of Cre-loxP recombination (Fig. S3A). Also, unlike the PA-Cre-M system, inactive TamPA-Cre is designed to be insensitive to non-specific light stimulation from, for example, the ambient white fluorescent lighting found in a typical lab or clinical environment. To restore its light sensitivity, TamPA-Cre must be primed by 4-OHT, which drives ERT2-CreN-nMag protein nuclear translocation. With both ERT2-CreN-nMag and NLS-pMag-CreC protein halves residing in the nucleus, blue light stimulation induces nMag-pMag heterodimerization, bringing CreN and CreC protein halves together to reconstitute the Cre recombinase activity of active TamPA-Cre. In this case, active TamPA-Cre initiates the expression of α-CD19 CAR in place of the anchoring myc-α-CD38 Receptor.

### Testing Photoactivatable Split-Cre Systems

To develop the optogenetic circuit, we first tested the two most recently developed blue light photoactivatable split-Cre systems. The PA-Cre-2.0 system (hereafter referred to as PA-Cre-C) relies on the blue-light-induced heterodimerization between CIB1 and CRY2(L348F) domains (Arabidopsis thaliano, heterodimer half-life: 24 min) to reconstruct functional Cre recombinase from the split CreN(19-104) and CreC(106-343) components.³⁰ The PA-Cre system (hereafter referred to as PA-Cre-M) utilizes the Neurospora crassa-derived Vivid photoreceptor mutant heterodimers negative Magnet (nMag) and positive Magnet (pMag) to reconstitute its split CreN(19-59) and CreC(60-343) components into functional Cre with blue light (heterodimer half-life: 1.8 hours).²⁹ To compare these two systems (Fig. S1A-C), we developed a puromycin-selected HEK293T cell line that stably expresses the Cre-loxP deletion-based EGFP Reporter construct (Fig. 2A, S1J) . We also built a novel controllable blue light stimulation apparatus that uses a diffusion filter to distribute light relatively evenly across a standard cell culture plate within a mammalian cell culture incubator (Fig. S2).

To compare the two photoactivatable split-Cre systems, EGFP Reporter HEK293T cells were or were not (Reporter) transiently transfected with either PA-Cre-C, PA-Cre-M, or Cre constructs (Fig. S1A-D). Then, cells either were (Light) or were not (Dark) briefly stimulated with a single pulse of blue light (472±29 nm, 30 W/m2, 30 s). The percentage of EGFP⁺ recombined cells in each cell group was determined by flow cytometry 24 h later. All EGFP Reporter HEK293T cell groups were normalized to the mean EGFP⁺ percentage of cells in the maximally recombined corresponding Cre⁺ positive control groups to account for differences in transfection efficiency between experiments.

As can be seen in FIG. 2B, EGFP Reporter HEK293T cells in both Light PA-Cre-C and Light PA-Cre-M groups responded to blue light stimulation with the expected increase in Cre-loxP recombination compared to their respective Dark counterparts (a 2.1- and 3.4-fold increase, respectively). The PA-Cre-M groups exhibited near maximal light-induced Cre-loxP recombination (90.8±9.0%, mean±standard deviation), whereas the Light PA-Cre-C groups reached only a fraction of that (12.1±2.2%). While different blue light intensities and patterns of exposure may have improved Cre-loxP recombination levels in the Light PA-Cre-C group, we prioritized robust recombination efficiency driven by minimal blue light energy. Therefore, we decided to pursue the highly sensitive, efficient, and robust nMag-CreN59 and pMag-CreC60 heterodimerizing domains of the PA-Cre-M system in our design.

However, despite the high recombination efficiencies of the Light PA-Cre-M groups, the Dark PA-Cre-M groups suffered from high background levels of spontaneous Cre-loxP recombination (26.7±2.0%), which was found to be proportional to PA-Cre-M protein expression levels (Fig. S3A). Furthermore, while stimulating the PA-Cre-M system in EGFP Reporter HEK293T cells with a pulsatile light protocol intended to improve its performance (15 W/m², is per min, 24 h), we found it necessary to actively protect non-blue-light-stimulated (Dark) cell groups from all light sources. Without protection, a small but significant additional percentage of cells underwent Cre-loxP recombination (Ambient: 28.9±4.8%, Dark: 20.4±2.4%), presumably driven by incidental exposure to the blue wavelengths of the laboratory's ambient white room lighting while in the incubator (0.34-0.78 W/m², FIG. 2C). Taken together, these results indicated that the highly photo-sensitive PA-Cre-M system was susceptible to leaky Cre-loxP recombination, which we next sought to minimize without sacrificing the system's light-driven high recombination efficiency.

### Testing Cre-ERT2 Systems

We focused on suppressing background Cre-loxP recombination in non-stimulated cells by spatially segregating one of the PA-Cre-M heterodimer split-Cre protein halves outside of the nucleus, since neither nuclear localization sequence (NLS) tagged CreN-nMag-NLS nor NLS-pMag-CreC protein halves alone were able to drive significant recombination (Fig. S1C, S3B). Although this could be achieved in a number of different ways (for example using the AsLOV2-based blue-light-inducible nuclear localization signal system),³¹ it was preferable to find an orthogonally-inducible, well-gated, and robust system compatible with use in vivo. The US Food and Drug Administration-approved drug tamoxifen and its active metabolites have been widely used to induce nuclear translocation of proteins, including Cre, which are fused to the T2 mutant Estrogen Receptor ligand binding domain (ERT2),³²⁻³⁴ particularly to induce genomic changes in vivo in transgenic mouse models.³⁵

Because tamoxifen and its active metabolites are known to be somewhat photosensitive,²² we examined whether or not relevant amounts of blue light stimulation interfered with the tamoxifen-gated ERT2-Cre-ERT2 Cre-loxP recombination system (FIG. 2D, S1E).³⁶ Applying the same pulsatile light stimulation pattern used in FIG. 2C, we found that tamoxifen metabolite 4-hydroxytamoxifen (4-OHT, 500 nM) stimulated robust Cre-loxP recombination efficiency in ERT2-Cre-ERT2⁺ EGFP Reporter HEK293T cells, surpassing the efficiency of the Cre⁺ positive control group despite blue light illumination (Dark: 127.0±15.6%, Ambient: 132.4±12.4%, Light: 116.1±9.0%). Furthermore, cell groups lacking 4-OHT stimulation exhibited minimal background recombination (Dark: 6.1±0.4%, Ambient: 6.5±0.7%, Light: 6.5±0.8%), highlighting that 4-OHT functions independently of the tested blue light stimulation parameters.

Tamoxifen-induced nuclear import translocation dynamics in HEK293T cells were further characterized using an ERT2-mCherry fusion protein (Fig. S1F). Using time-lapse fluorescence microscopy, we discovered that nuclear translocation driven by 4-OHT (500 nM) occurred on the order of hours. ERT2-mCherry protein was only clearly nuclear-localized (with a nuclear-to-cytosolic mean fluorescence intensity ratio≥2) after approximately three hours (FIG. 2E), highlighting an effective time window for blue light stimulation to drive robust Cre-loxP recombination in ERT2-based photoactivatable construct designs.

### Design and Function of TamPA-Cre

We therefore integrated components of both the Tamoxifen-ERT2 and Photoactivatable-Cre systems to create a novel genetically-encoded AND gate in which both tamoxifen and blue light stimulation are needed to drive Cre-loxP recombination (FIG. 3A). In our system, named TamPA-Cre, the ERT2 domain was fused to the smaller CreN59-nMag construct (Fig. S1H1), and not to pMag-CreC60 (Fig. S1C2) which contains potentially competitive NLSs native to Cre. The TamPA-Cre system's heterodimers were designed to be expressed at similar levels, like the PA-Cre-M system, with both proteins translated from a single transcript and separated by the P2A self-cleaving peptide (Fig. S1B). The ERT2-CreN(2-59) sequence was fused to the N-terminus of nMag (without NLS), and fluorescent marker P2A-mCherry was added to the C-terminus of NLS-pMag-CreC60 to confirm expression. Our design also included a codon-diversified pMag coding sequence (CDS) to prevent potential recombination introduced by lentiviral gene transfer between the nearly identical nMag and pMag CDSs (Fig. S4).³⁷ Although this construct worked well for transient expression, its large single transcript size significantly impeded lentivirus production efficiency. Therefore, the ERT2-CreN-nMag and NLS-pMag-CreC components were separated into two vectors, each with a unique fluorescent marker (Fig. S1H). The similarity in transcript size of ERT2-CreN-nMag and NLS-pMag-CreC (approximately 1.6 and 1.4 kb, respectively) resulted in similar protein expression levels and comparable efficiency in lentivirus production. We additionally created ERT2-CreN-nMagHigh1, which contains the mutated nMag variant nMagHigh1 (M135I/M165I) previously shown to improve light-induced heterodimerization with pMag.³⁸ Along with NLS-pMag-CreC, this system is referred to as TamPA-Cre-nH1 (Fig. S1I).

The mechanism of TamPA-Cre activation is illustrated in FIG. 3B. Without tamoxifen- or blue light stimulation, TamPA-Cre is inactive with ERT2-CreN-nMag and NLS-pMag-CreC proteins spatially segregated to the cytosol and nucleus, respectively. This physical separation prevents the spontaneous, concentration-dependent nMag-pMag dimerization seen in the PA-Cre-M system and thus suppresses the unwanted background levels of Cre-loxP recombination (Fig. S3A). Also, unlike the PA-Cre-M system, inactive TamPA-Cre is designed to be insensitive to non-specific light stimulation from, for example, the ambient white fluorescent lighting found in a typical lab or clinical environment. To restore its light sensitivity, TamPA-Cre must be primed by 4-OHT, which drives ERT2-CreN-nMag protein nuclear translocation. With both ERT2-CreN-nMag and NLS-pMag-CreC protein halves residing in the nucleus, blue light stimulation induces nMag-pMag heterodimerization, bringing CreN and CreC protein halves together to reconstitute the Cre recombinase activity of active TamPA-Cre.

### Optimizing Tamoxifen and Blue Light Stimulation

To optimize Cre-loxP recombination, we tested the TamPA-Cre system in EGFP Reporter HEK293T cells with a variety of tamoxifen and blue light stimulation protocols. Two parameters were found to be particularly important: the light stimulation pattern (pulsatile versus continuous exposure), and the time at which light was started relative to 4-OHT addition. Drawn from the 3 h short illumination pattern shown to improve PA-Cre-M function,²⁹ FIG. 3C illustrates two different tamoxifen and light stimulation protocols that each call for a total three hours of 5 W/m2 blue light stimulation. In Protocol A, light is applied continuously with concurrent 4-OHT addition. In Protocol B, 4-OHT is added three hours prior to delivering light in an even pulsatile pattern over the course of 24 h (FIG. 3C).

Under Protocol A, light-stimulated TamPA-Cre drove Cre-loxP recombination in only a minor percentage of EGFP Reporter HEK cells (17.3±1.1%, FIG. 3D). However, TamPA-Cre-mediated recombination was improved when the start of light stimulation was delayed by several hours relative to 4-OHT addition (24.6±5.1%, Fig. S5A). Considering that the 4-OHT-stimulated ERT2-mCh fusion protein reached significant 4-OHT-induced nuclear translocation only after approximately 3 h (FIG. 2E), the poor function of TamPA-Cre under Protocol A is likely due to suboptimal nuclear ERT2-CreN-nMag concentrations.

TamPA-Cre-mediated recombination was also improved to a similar extent by administering light in a pulsatile pattern started concurrently with tamoxifen stimulation (25.4±3.5%, Fig. S5B). p Therefore, Protocol B was created to merge the advantages of both a delay in light stimulation and a pulsatile light pattern. In EGFP Reporter HEK293T cells exposed to the same total three hours of blue light stimulation but delivered in a pulsatile pattern (7.5 s per min, 24 h) started three hours post-tamoxifen stimulation (Protocol B), TamPA-Cre drove robust levels of Cre-loxP recombination (79.9±10.1%, FIG. 3D)—on par with the performance of PA-Cre-M (84.8±3.1%, Fig. S5C). Moreover, the PA-Cre-M system's high levels of non-light-stimulated background Cre-loxP recombination (Dark: 32.9±6.3%, Ambient: 45.2±8.1%) were significantly suppressed by the AND gate of the TamPA-Cre system (Dark: 9.6±2.8%, Ambient: 10.3±0.3%). The AND gate also successfully suppressed blue light-stimulated recombination in the absence of 4-OHT (15.4±2.3%).

With the AND gate working as intended, we further investigated whether the reported 13-fold increase in heterodimerization between the pMag and nMagHigh1 domains³⁸ would additionally improve recombination efficiency as TamPA-Cre-nH1 (Fig. S1I). Under Protocol B, a greater percentage of TamPA-Cre-nH1⁺ EGFP Reporter HEK cells did indeed undergo Cre-loxP recombination (125.0±13.3%, FIG. 3E), but at the cost of an increase in background recombination in non-blue-light-stimulated cell groups (Dark: 21.8±1.5%, Ambient: 34.6±3.2%). The AND gate of TamPA-Cre-nH1 also failed to sufficiently suppress light-induced recombination in the absence of 4-OHT (50.7±5.9%). The TamPA-Cre system was thus a safer choice for us to use in reaching our goal to strictly regulate localized CAR expression in T cells to avoid on-tumor off-target toxicities.

Design and function of the CAR Reporter

The TamPA-Cre system was next applied to induce CAR expression in the physiologically relevant Jurkat T cell line. To demonstrate our overall concept outlined in FIG. 1, CD19 and CD38 TAAs were targeted with an α-CD19 CAR and a c-myc-tagged α-CD38 Receptor, respectively (Fig. S6A).³⁹ A Jurkat T cell line stably expressing the α-CD19CAR-EGFP was verified to undergo CD19-specific CAR-mediated T cell activation, as measured by upregulated CD69 expression. Co-incubation with CD19⁺ Toledo Target cells activated Jurkat CART cells (81.1±0.35%), whereas co-incubation with CD19-K562 Target cells did not (3.18±0.09%, Fig. S6B,D). Likewise, Jurkat T cells stably expressing myc-α-CD38 Receptor were not activated upon binding to CD38⁺ Toledo Target cells, as expected (Fig. S6D).

The CAR and Receptor constructs were next integrated into several different foxed reporter designs to achieve the initial expression of the tumor-anchoring myc-α-CD38 Receptor, and α-CD19 CAR expression only upon induction via TamPA-Cre-mediated Cre-loxP recombination. After testing the TamPA-Cre system with several different reporter configurations (Fig. S7), the deletion-based Cre-loxP recombination CAR Reporter was selected to create the CAR Reporter Jurkat T cell line (FIG. 4A, S1K). As CAR over-expression can lead to spontaneous T cell activation (Fig. S6D), this cell line was transduced with a single copy (on average) of the CAR Reporter. The CAR Reporter consists of an hEF1α promoter driving the expression of foxed myc-α-CD38Receptor (with stop codons to halt translation), followed by α-CD19CAR-EGFP. The Receptor is designed to allow patient-administered CAR T cells to home in and bind to CD38⁺ cells, both cancerous and healthy, in vivo. When the cancerous region is locally stimulated with blue light following global tamoxifen administration, TamPA-Cre excises myc-α-CD38Receptor from the genome (along with its stop codons), allowing for rapid α-CD19CAR-EGFP expression and subsequent T cell effector functions (FIG. 4B,E).

Both components of either the PA-Cre-M or TamPA-Cre system were transduced into CAR Reporter Jurkat T cells sequentially at high copy number and maintained via puromycin selection to create stable cell lines. CAR Reporter Jurkat T cells transduced with only one of the two TamPA-Cre components were unable to undergo Cre-loxP recombination indicating that, like PA-Cre-M, both split-Cre protein halves are necessary for function (Fig. S6E). The PA-Cre-W and TamPA-Cre⁺ CAR Reporter Jurkat T lines were protected from light whenever possible during cell line development and culture to minimize potential ambient light-driven background recombination.

In order to preemptively address reported blue light phototoxicity concerns in T cells,⁴⁰ we further reduced the total amount of blue light stimulation time from 3 h in Protocol B to 2 h in Protocol E (5 W/m2, 5 s per min, 24 h, FIG. 4C). This reduction was possible due to the slow dissociation kinetics of the PA-Cre-M system's nMag-pMag heterodimerized proteins (half-life: 1.8 h).³⁸ Assuming stimulated TamPA-Cre heterodimerized proteins dissociate similarly upon the removal of light, less than 1% of active TamPA-Cre proteins will have been lost to dissociation during the 52.5 s or 55 s of darkness between the blue light pulses of Protocol B and E, respectively (Fig. S8A). In essence, the pulsatile light pattern uses minimal light energy to maintain near-maximal levels of tamoxifen-primed active TamPA-Cre expression (Fig. S8B). Three hours after incubation with 4-OHT, TamPA-Cre⁺ CAR Reporter Jurkat T cells were subjected to Protocol E's 5 s per min pulsatile blue light stimulation pattern for 0, 1, 3, 6, or 24 hours, after which the percentage of CAR-EGFP⁺ recombined cells in each group was determined by flow cytometry 48 h after the start of light exposure (FIG. 4D). A positive control Cre⁺ CAR Reporter Jurkat T cell line with comparable protein expression levels could not be established due to long-term cytotoxicity. Therefore, the percentage of recombined CAR-EGFP⁺ cells in each CAR Reporter Jurkat T cell group was normalized to its mean maximal recombination measured from the initial percentage of myc-α-CD38 Receptor⁺ cells capable of undergoing recombination in each group. Cre-loxP recombination efficiency increased exponentially with the total duration of blue light stimulation, and plateaued within 24 hours of exposure (Fig. S9, Table S2).

Following Protocol E with 24 h of blue light stimulation, the percentage of TamPA-Cre⁺ CAR Reporter Jurkat T cells expressing myc-α-CD38 Receptor and α-CD19 CAR-EGFP was tracked over several days (FIG. 4E, Table S2). Without a means to replenish receptor proteins after the myc-α-CD38Receptor CDS is excised during Cre-loxP recombination, the fraction of Receptor⁺ cells decayed exponentially over time (half-life: ˜33.7 h), reaching a minimum of 9.3%±1.3% five days after the start of blue light stimulation. Simultaneously, the percentage of CAR⁺ cells increased exponentially (half-life: 13.3 h), more rapidly than the decrease in myc-α-CD38 Receptor expression. As such, engineered TamPA-Cre CAR Reporter T cells should remain anchored as locally-applied blue light triggers CAR expression for tumor eradication (FIG. 5A).

An exemplary nucleic acid sequence for an exemplary EF1α promoter is:

(SEQ ID NO: 7)
Ggctccggtgcccgtcagtgggcagagcgcacatcg
cccacagtccccgagaagttggggggaggggtcgg
caattgaaccggtgcctagagaaggtggcgcgggt
aaactgggaaagtgatgtcgtgtactggctccgcc
tttttcccgagggtgggggagaaccgtatataagt
gcagtagcgccgtgaacgttctttttcgcaacggg
tttgccgccagaaacacaggtaagtgccgtgtgtg
gttcccgcgggccttggcctctttacgggttatgg
cccttgcgtgccttgaattacttccacctggctgc
agtacgtgattcttgatcccgagcttcgggttgga
agtgggtgggagagttcgaggccttgcgcttaagg
agccccttcgcctcgtgcttgagttgaaggcctgg
cctgggcgctggggccgccgccgcgtgcgaatctg
gtggcaccttcgcgcctgtctcgctgctttcgata
agtctctagccatttaaaatttttgatgacctgct
gcgacgctttttttctggcaagatagtcttgtaaa
tgcgggccaagatctgcacactggtatttcggttt
ttggggccgcgggcggcgacggggcccgtgcgtcc
cagcgcacatgttcggtgaggcggggcctgcgagc
gcggccaccgagaatcggacgggggtagtctcaag
ctggccggcctgctctggtgcctggcctcgcgccg
ccgtgtatcgccccgccctgggcggcaaggctggc
ccggtcggcaccagttgcgtgagcggaaagatggc
cgcttcccggccctgctgcagggagctcaaaatgg
aggacgcgggcgctcgggagagcgggcgggtgagt
cacccacacaaaggaaaagggcctttccgtcctca
gccgtcgcttcatgtgactccacggagtaccgggc
gccgtccaggcacctcgattagttctcgagctttt
ggagtacgtcgtcttaggttggggggaggggtttt
atgcgatggagttccccacactgagtgggtggaga
ctgaagttaggccagcttggcacttgatgtgtaat
tctccttggaatttgcccttttgagtttggatctt
ggttcattctcaagcctcagacagtggttcaaagt
ttttttcttccatttcaggtgtcgtga

An exemplary nucleic acid sequence for an exemplary myc-α-CD38-Receptor is:

(SEQ ID NO: 8)
Atggagtttggactgagctggttgttcctcgtagc
tattctgaagggcgttcagtgctctgagcaaaagt
tgatctcagaagaggacctggacatccagttgacc
cagagtagctcctccttctctgtgagcctgggaga
tcgggtgactattacctgtaaagcgtctgaagata
tttacaaccgcctggcgtggtaccagcagaaacca
ggaaacgccccaagacttctcatatctggagccac
tagccttgaaactggcgtaccctcccgattttccg
ggtccggaagtggcaaagattataccctctccatt
acctctcttcagacggaggacgtggcaacatacta
ttgtcaacagtactggtctacacctacctttggtg
ggggaaccaagctggaaatcaagagggccgatgcc
gctcccactgtttcaatcttcccaccctctggcgg
cggcgggagcggcggtggtggcagcgggggtggtg
ggtccggaggtggggggtcacaggtccagctgaag
caaagcggccctagtctcgtccagccctcacaaag
actgtccatcacctgtaccgtcagcggcttttctc
tgatcagctatggagtccattgggtgaggcaatct
cctggaaagggactggaatggctgggggtgatctg
gaggggcgggtccaccgattacaacgccgcattca
tgtcacgcctgtccatcactaaagacaactccaaa
tctcaagtgttctttaaaatgaactccctgcaggc
tgatgacaccgctatctacttctgtgcaaaaacgc
tgataactacgggttacgccatggattactggggc
cagggtacctccgtcaccgttagctctGCCGC
AGCTATCGAGGTCATGTACCCGCCACCCTATCT
CGATAACGAAAAATCCAACGGGACAATCATACACG
TAAAGGGCAAGCATTTGTGCCCCTCCCCTTTGTTC
CCAGGGCCGTCAAAACCATTCTGGGTCTTGGTAGT
CGTAGGAGGTGTGCTTGCTTGTTACTCACTGTTGG
TTACTCTTGCGTTCATAATCTTTTGGGTGCGGTCA
AAACGCTCC

An exemplary nucleic acid sequence for an exemplary α-CD19CAR-EGFP is:

(SEQ ID NO: 9)
ATGGCTCTCCCAGTGACTGCCCTACTGCTTCCCCT
AGCGCTTCTCCTGCATGCAGAGGTGAAGCTGCAGC
AGTCTGGGGCTGAGCTGGTGAGGCCTGGGTCCTCA
GTGAAGATTTCCTGCAAGGCTTCTGGCTATGCATT
CAGTAGCTACTGGATGAACTGGGTGAAGCAGAGGC
CTGGACAGGGTCTTGAGTGGATTGGACAGATTTAT
CCTGGAGATGGTGATACTAACTACAATGGAAAGTT
CAAGGGTCAAGCCACACTGACTGCAGACAAATCCT
CCAGCACAGCCTACATGCAGCTCAGCGGCCTAACA
TCTGAGGACTCTGCGGTCTATTTCTGTGCAAGAAA
GACCATTAGTTCGGTAGTAGATTTCTACTTTGACT
ACTGGGGCCAAGGGACCACGGTCACCGTCTCCTCA
GGTGGAGGTGGATCAGGTGGAGGTGGATCTGGTGG
AGGTGGATCTGACATTGAGCTCACCCAGTCTCCAA
AATTCATGTCCACATCAGTAGGAGACAGGGTCAGC
GTCACCTGCAAGGCCAGTCAGAATGTGGGTACTAA
TGTAGCTGGTATCAACAGAAACCAGGACAATCTCC
TAAACCACTGATTTACTCGGCAACCTACCGGAACA
GTGGAGTCCCTGATCGCTTCACAGGCAGTGCATCT
GGGACAGATTTCACTCTCACCATCACTAACGTGCA
GTCTAAAGACTTGGCAGAaATTTCTGTCAACAATA
TAACAGGTATCCGTACACGTCCGGAGGGGGGACCA
AGGCTGGAGATCAAACGGGCGGCCGCAattgaagt
tatgtatcctccttacctagacaatgagaagagca
atggaaccattatccatgtgaaagggaaacacctt
tgtccaagtcccctatttcccggaccttctaagcc
cttttgggtgctggtggtggttggtggagtcctgg
cttgctatagcttgctagtaacagtggcctttatt
attttctgggtgaggagtaagaggagcaggctcct
gcacagtgactacatgaacatgactccccgccgcc
ccgggcccacccgcaagcattaccagccctatgcc
ccaccacgcgacttcgcagcctatcgctccCGTTT
CTCTGTTGTTAAACGGGGCAGAAAGAACCTCCTGT
ATATATTCAAACAACCATTTATGAGACCAGTACAA
ACTACTCAAGAGGAAGATGGCTGTAGCTGCCGATT
TCCAGAAGAAGAAGAAGGAGGATGTGAACTGCTTT
ACTGCAACCACAGAGTGAAGTTCAGCAGGAGCGCA
GAGCCCCCCGCGTACCAGCAGGGCCAGAACCAGCT
CTATAACGAGCTCAATCTAGGACGAAGAGAGGAGT
ACGATGTTTTGGACAAGAGACGTGGCCGGGACCCT
GAGATGGGGGGAAAGCCGAGAAGGAAGAACCCTCA
GGAAGGCCTGTACAATGAACTGCAGAAAGATAAGA
TGGCGGAGGCCTACAGTGAGATTGGGATGAAAGGC
GAGCGCCGGAGGGGCAAGGGGCACGATGGCCTTTA
CCAGGGTCTCAGTACAGCCACCAAGGACACCTACG
ACGCCCTTCACATGCAGGCCCTGCCCCCTCGCGGT
GGAAGTGGCGGTACCATGGTGAGCAAGGGCGAGGA
GCTGTTCACCGGGGTGGTGCCCATCCTGGTCGAGC
TGGACGGCGACGTAAACGGCCACAAGTTCAGCGTG
TCCGGCGAGGGCGAGGGCGATGCCACCTACGGCAA
.GCTGACCCTGAAGTTCATCTGCACCACCGGCAAC
TCTGCCCGTGCCCTGGCCCACCCTCGTGACCACCC
TGACCTACGGCGTGCAGTGCTTCAGCCGCTACCCC
GACCACATGAAGCAGCACGACTTCTTCAAGTCCGC
CATGCCCGAAGGCTACGTCCAGGAGCGCACCATCT
TCTTCAAGGACGACGGCAACTACAAGACCCGCGCC
GAGGTGAAGTTCGAGGGCGACACCCTGGTGAACCG
CATCGAGCTGAAGGGCATCGACTTCAAGGAGGACG
GCAACATCCTGGGGGCACAAGCTGGAGTACAACTA
CAACAGCCACAACGTCTATATCATGGCCGACAAGC
AGAAGAACGGCATCAAGGTGAACTTCAAGATCCGC
CACAACATCGAGGACGGCAGCGTGCAGCTCGCCGA
CCACTACCAGCAGAACACCCCCATCGGCGACGGGC
CCGTGCTGCTGCCCGACAACCACTACCTGAGCACC
CAGTCCGCCCTGAGCAAAGACCCCAACGAGAAGCG
CGATCACATGGTCCTGCTGGAGTTCGTGACCGCCG
CCGGGATCACTCTCGGCATGGACGAGCTGTACAAG

An exemplary nucleic acid sequence for an exemplary CAR Reporter (EF1α promoter-LoxP-myc-α-CD30-Receptor-LoxP-α-CD19CAR-EGFP) is:

(SEQ ID NO: 10)
ggctccggtgcccgtcagtgggcagagcgcacatc
gcccacagtccccgagaagttggggggaggggtcg
gcaattgaaccggtgcctagagaaggtggcgcggg
gtaaactgggaaagtgatgtcgtgtactggctccg
cctttttcccgagggtgggggagaaccgtatataa
gtgcagtagtcgccgtgaacgttctttttcgcaac
gggtttgccgccagaacacaggtaagtgccgtgtg
tggttcccgcgggcctggcctctttacgggttatg
gcccttgcgtgccttgaattacttccacctggctg
cagtacgtgattcttgatcccgagcttcgggttgg
aagtgggtgggagagttcgaggccttgcgcttaag
gagccccttcgcctcgtgcttgagttgaggcctgg
cctgggcgctggggccgccgcgtgcgaatctggtg
gcaccttcgcgcctgtctcgctgctttcgataagt
ctcagccatttaaaattttgatgacctgctgcgac
gcttttttctggcaagatagtcttgtaaatgcggg
ccaagatctgcacactggtatttcggtttttgggg
ccgcgggcggcgacggggcccgtgcgtcccagcgc
acatgttcggcgaggcggggcctgcgagcgcggcc
accgagaatcggacgggggtagtctcaagctggcc
ggcctgctctggtgcctggcctcgcgccgccgtgt
atcgccccgccctgggcggcaaggctggcccggtc
ggcaccagttgcgtgagcggaaagatggccgcttc
ccggcctgctgcagggagctcaaaatggaggacgc
ggcgctcgggagagcgggcgggtgagtcacccaca
caaaggaaaagggcctttccgtcctcagccgtcgc
ttcatgtgactccacggagtaccgggcgccgtcca
ggcacctcgattagttctcgaagcttttggagtac
gtcgtcttaggttggggggaggggttttatgcgat
ggagtttccccacactgagtgggtggagactgaag
ttaggccagcttggcacttgatgtaattctccttg
gaattgcccttttgagtttggatcttggttcattc
tcaagcctcagacagtggttcaaagtttttttctt
ccatttcaggtgtcgtgaggaattgatccttcgaa
ctagtttctgcagtcgacCGGATTATAACTTCGTA
TAGCATACATTATACGAAGTTATAATTCTGCAGAT
ATCCAGCACAGTGGCGGCCACCatggagtttggac
tgagctggttgttcctcgtagctattctgaagggc
gttcagtgctctgagcaaaagttgatctcagaaga
ggacctggacatccagttgacccagagtagctcct
ccttctctgtgagcctgggagatcgggtgact&tt
acetgtaaagcgtctgaagatatttacaacegcct
ggcgtggtaccagc&gaaaccaggaaacgccccaa
gacttctcatatctggagccactagccttgaaact
ggcgtaccctcccgattttccgggtccggaagtgg
caaagtggggggaaccaagctggaaatcaagaggg
ccgatgccgctcccactgtttcaatcttcccaccc
tctggcggcggcgggagcggcggtggtggcagcgg
gggtggtgggtccggaggtggggggtcacaggtcc
agtagaagcaaagcggccctagtctcgtccagccc
tcacaaagactgtccatcacctgtaccgtcagcgg
cttttctctgatcagctatggagtccattgggtga
ggcaatctcctggaaagggactggaatggctgggg
gtgatctggaggggcgggtccaccgattacaacgc
cgcattcatgtcacgcctgtccatcactaaagaca
actccaaatctcaagtgttctttaaaatgaactcc
ctgcaggctgatgacaccgctatctacttctgtgc
aaaaacgctgataactacgggttacgecatggatt
actggggccagggtacctccgtcaccgttagctct
GCCGCAGCTATCGAGGTCATGTACCCGCCACCCTA
TCTCGATAACGAAAAATCCAACGGGACAATCATAC
ACGTAAAGGGCAAGCATTTGTGCCCCTCCCCTTTG
TTCCCAGGGCCGTCAAAACCATTCTGGGTCTTGGT
AGTCGTAGGAGGTGTGCTTGCTTGTTACTCACTGT
TGGTTACTGTTGCGTTCATAATCTTTTGGGTGCGG
TCAAAACGCTCCtgataagctagcTGAATAAGGCC
GCTCGAATAACTTCGTATAGCATACATTATACGAA
GtTAXGTTTcgggAATTCGATGGCTCTCCCAGTGA
CTGCCCTACTGCTTCCCCTAGCGCTTCTCCTGCAT
GCAGAGGTGAAGCTGCAGCAGTCTGGGGCTGAGCT
GGTGAGGCCTGGGTCCTCAGTGAAGATTTCCTGGA
AGGCTTCTGGCTATGCATTCAGTAGCTACTGGATG
AACTGGGTGAAGCAGAGGCCTGGACAGGGTCTTGA
GTGGATTGGACAGATTTATCCTGGAGATGGTGATA
CTAACTACAATGGAAAGTTCAAGGGTCAAGCCACA
CTGACTGCAGACAAATCCTCCAGCACAGCCTACAT
GGAGCTCAGCGGCCTAACATCTGAGGACTCTGCGG
TCTATTTCTGTGCAAGAAAGACCATTAGTTCGGTA
GTAGATTTCTACTTTGACTACTGGGGCCAAGGACC
ACGGTCACCGTCTCCTCAGGTGGAGGTGGATCAGG
TGGAGGTGGATCTGGTGGAGGTGGATCTGACATTG
AGCTCACCCAGTCTCCAAAATTCATGTCCACATCA
GTAGGAGACAGGGTCAGCGTCACCTGCAAGGCCAG
TCAGAATGTGGGTACTAATGTAGCCTGGTATCAAC
AGAAACCAGGACAATCTCCTAAACCACTGATTTAC
TCGGCAACCTACCGGAACAGTGGAGTCCCTGATCG
CTTCACAGGCAGTGGATGTGGGACAGATTTCACTC
TCAGGATCACTAACGTGCAGTCTAAAGACTTGGCA
GACTATTTCTGTCAACAATATAACAGGTATCCGTA
CACGTCCGGAGGGGGGACCAAGCTGGAGATCAAAC
GGGCGGCCGCAattgaagttatgtatcctcctcct
tacctagacaatgagaagagcaatggaaccattat
ccatgtgaaagggaaacacctttgtccaagtcccc
tatttcccggaccttctaagcccttttgggtgctg
gtggtggttggtggagtcctggcttgctatagctt
gctagtaacagtggcctttattattttctgggtga
ggagtaagaggagcaggctcctgcacagtgactac
atgaacatgactccccgccgccccgggcccacccg
caagcattaccagccctatgccccaccacgcgact
tcgcagcctatcgctccCGTTTCTCTGTTGTTAAA
CGGGGCAGAAAAGAAGCTCCTGTATATATTCAAAC
AACCATTTATGAGACCAGTACAAACTACTCAAGAG
GAAGATGGCTGTAGCTGCCGATTTCCAGAAGAAGA
AGAAGGAGGATGTGAACTGCTTTACTGCAACCACA
GAGTGAAGTTCAGCAGGAGCGCAGAGCCCCCCGCG
TACCAGCAGGGCCAGAACCAGCTCTATAACGAGCT
CAATCTAGGACGAAGAGAGGAGTACGATGTTTTGG
ACAAGAGACGTGGCCGGGACCCTGAGATGGGGGGA
AAGCCGAGAAGGAAGAACCCTCAGGAAGGCCTGTA
CAATGAACTGCAGAAAGATAAGATGGCGGAGGCCT
ACAGTGAGATTGGGATGAAAGGCGAGCGCCGGAGG
GGCAAGGGGCACGATGGCCTTTACCAGGGTCTCAG
TACAGCCACCAAGGACACCTACGACGCCCTTCACA
TGCAGGCCCTGCCCCCTCGCGGTGGAAGTGGCGGT
ACCATGGTGAGCAAGGGCGAGGAGCTGTTCACCGG
GGTGGTGCCCATCCTGGTCGAGCTGGACGGCGACG
TAAACGGCCACAAGTTCAGCGTGTCCGGCGAGGGC
GAGGGCGATGCCACCTACGGCAAGCTGACCCTGAA
GTTCATCTGCACCACCGGCAAGCTGCCCCGTGCCC
TGGCCCACCCTCGTGACCACCCTGACCTACGGCGT
GCAGTGCTTCAGCCGCTACCCCGACCACATGAAGC
AGCACGACTTCTTCAAGTCCGCCATGCCCGAAGGC
TACGTCCAGGAGCGCACCATCTTCTTCAAGGACGA
CGGCAACTACAAGACCCGCGCCGAGGTGAAGTTCG
AGGGCGACAGGGTGGTGAACCGCATCGAGCTGAAG
GGCATCGACTTCAAGGAGGACGGCAACATCCTGGG
GCACAAGCTGGAGTACAACTACAACAGCCACAACG
TCTATATCATGGCCGACAAGCAGAAGAACGGCATC
AAGGTGAACTTCAAGATCCGCCACAACATCGAGGA
CGGCAGCGTGCAGCTCGCCGACCACTACCAGCAGA
ACACCCCATCGGCGACGGCCCCGTGCTGCTGCCCG
ACAACCACTACCTGAGCACCCAGTCCGCCCTGAGC
AAAGACCCCAACGAGAAGCGCGATCACATGGTCCT
GCTGGAGTTCGTGACCGCCGCCGGGATCACTCTCG
GCATGGACGAGCTGTACAAGtaatag

In alternative embodiments, although the exemplary construct uses the linkers:

(SEQ ID NO: 11)
tgataagctagc,
and,
(SEQ ID NO: 12)
GTTTcgggAATTCG

any linker known in the art can be used in place of SEQ ID NO:11 or SEQ ID NO:12, there is no specific minimum or maximum length for a linker that can be used in constructs as provided herein, and there is no specific sequence or structural requirement for any linker that can be used in this or any construct as provided herein.

In alternative embodiments, although the exemplary construct uses the stop codon cassette sequence: TGAATAAGGCCGCTCGA (SEQ ID NO:13), alternative stop codon cassettes can be used, for example, codons or cassettes that comprise TAA, TAG or TGA stop codons.

In alternative embodiments, although the exemplary construct comprises use of the two stop codons taatag at the end of the construct's sequence (to signal the termination of the translation process of the encoded protein), any stop codon cassette comprising taa or tga or tag can be used; for example, in alternative embodiments, equivalent sequences for tatag can be any two combination of taa or tga or tag, for example, such as taataa, taatga, and/or taatag.

### TamPA-Cre Drives CAR-Mediated T Cell Activation

In a head-to-head comparison, CAR Reporter Jurkat T cell lines expressing TamPA-Cre, PA-Cre-M, or neither (Reporter) were (Light) or were not (Dark) subjected to tamoxifen and/or blue light stimulation in accordance with Protocol E. Two days after the start of light stimulation, each group of cells either were (+Target) or were not (-Target) co-cultured for 24 h with an equal number of CD19⁺ Toledo Target cells. All groups were then analyzed 24 h later for the expression of α-CD19 CAR-EGFP and the early T cell activation marker CD69 via flow cytometry. The percentage of recombined CAR-EGFP⁺ cells and the percentage of activated CD69⁺ cells in each CAR Reporter Jurkat T cell group were normalized to maximal recombination calculated from the initial percentage of myc-α-CD38 Receptor⁺ cells capable of undergoing Cre-loxP recombination.

In a trend consistent with HEK293T experiments, light stimulation drove a significant 4.1-fold increase in the normalized percentage of recombined PA-Cre-M⁺ CAR Reporter Jurkat T cells (Light: 21.2±1.9%, Dark: 5.2±0.7%), whereas tamoxifen- and blue light-stimulated TamPA-Cre⁺ cells exhibited a robust 27.1-fold increase (Light+4-OHT: 52.4±1.5%, Dark+4-OHT: 1.9±0.5%, FIG. 5B-C, S10A-B). Upon co-culture with CD19⁺ Target cells, light-stimulated PA-Cre-M⁺ groups exhibited activation in less than half of CAR Reporter Jurkat T cells—a 4.4-fold increase compared to those without light stimulation (40.6±3.5% and 9.3±1.3%, respectively, FIG. 5D, S10C). On the other hand, tamoxifen- and blue-light-stimulated TamPA-Cre drove CAR-mediated T cell activation in nearly all CAR Reporter Jurkat T cells co-cultured with CD19⁺ Target cells (92.7±2.4%)—a 22.3-fold increase from those groups lacking light stimulation (4.2±0.1%, FIG. 5E-F, S10D). Furthermore, without tamoxifen, blue light stimulation drove only a minor increase in TamPA-Cre-mediated Cre-loxP recombination and activation (4.4±0.8% and 7.6±0.8%, respectively), indicating that the AND gate functions as intended in Jurkat T cells.

While the suppression of partially-stimulated cells was not perfect, the TamPA-Cre system's high recombination efficiency upon complete stimulation makes it amenable to further means of suppression (for example lowering TamPA-Cre expression, additional gating, etc.) Moreover, exposing TamPA-Cre⁺ CAR Reporter Jurkat T cells to 48 h of ambient light did not drive any additional background recombination, unlike PA-Cre-M⁺ cells (Fig. S6F). Therefore, without tamoxifen stimulation, cells expressing the TamPA-Cre system are relatively safe from background CAR expression—a requirement for practical applications. As such, with robust and well-gated tamoxifen- and blue light-inducible CAR expression and T cell activation, the TamPA-Cre system proves to be an effective tool with which to control localized CAR-mediated T cell activation.

### REFERENCES

1. Wu, C. Y., et al., Remote control of therapeutic T cells through a small molecule-gated chimeric receptor. Science 2015, 350 (6258).
1. Wu, C. Y., et al., Remote control of therapeutic T cells through a small molecule-gated chimeric receptor. Science 2015, 350 (6258).
2. Kloss, C. C., et al., Combinatorial antigen recognition with balanced signaling promotes selective tumor eradication by engineered T cells. Nat Biotechnol 2013, 31 (1), 71-+.
3. Roybal, K. T., et al., Precision Tumor Recognition by T Cells With Combinatorial Antigen-Sensing Circuits. Cell 2016, 164 (4), 770-9.
4. Taslimi, A., et al., Optimized second-generation CRY2-CIB dimerizers and photoactivatable Cre recombinase. Nat Chem Biol 2016, 12 (6), 425-30.
5. Kawano, F., et al., A photoactivatable Cre-loxP recombination system for optogenetic genome engineering. Nat Chem Biol 2016, 12 (12), 1059-+.
6. Komatsubara, A. T.; Matsuda, M.; Aoki, K., Quantitative analysis of recombination between YFP and CFP genes of FRET biosensors introduced by lentiviral or retroviral gene transfer. Sci Rep 2015, 5, 13283.
7. Kawano, F.; Suzuki, H.; Furuya, A.; Sato, M., Engineered pairs of distinct photoswitches for optogenetic control of cellular proteins. Nat Commun 2015, 6, 6256.
8. Cho, J. H.; Collins, J. J.; Wong, W. W., Universal Chimeric Antigen Receptors for Multiplexed and Logical Control of T Cell Responses. Cell 2018.
9. Di Stasi, A.; Tey, S. K.; Dotti, G.; Fujita, Y.; Kennedy-Nasser, A.; Martinez, C.; Straathof, K.; et al., Inducible Apoptosis as a Safety Switch for Adoptive Cell Therapy. New Engl J Med 2011, 365 (18), 1673-1683.
10. Gargett, T.; Brown, M. P., The inducible caspase-9 suicide gene system as a “safety switch” to limit on-target, off-tumor toxicities of chimeric antigen receptor T cells. Front Pharmacol 2014, 5, 235.
11. Fedorov, V. D.; et al., PD-1-and CTLA-4-Based Inhibitory Chimeric Antigen Receptors (iCARs) Divert Off-Target Immunotherapy Responses. Sci Transl Med 2013, 5 (215).
12. Eyquem, J.; et al., Targeting a CAR to the TRAC locus with CRISPR/Cas9 enhances tumour rejection. Nature 2017, 543 (7643), 113-117.
13. Kristianto, J., et al., Spontaneous recombinase activity of Cre-ERT2 in vivo. Transgenic Res 2017, 26 (3), 411-417.
14. Niopek, D., et al., Engineering light-inducible nuclear localization signals for precise spatiotemporal control of protein dynamics in living cells. Nature Communications 2014, 5.
15. Feil, R.; Wagner, J.; Metzger, D.; Chambon, P., Regulation of Cre recombinase activity by mutated estrogen receptor ligand-binding domains. Biochem Bioph Res Co 1997, 237 (3), 752-757.
16. Hirrlinger, J.; et al., Split-CreERT2: Temporal Control of DNA Recombination Mediated by Split-Cre Protein Fragment Complementation. Plos One 2009, 4 (12).
17. Besser, S., et al., Split-Cre and Split-Creert2: Versatile Genetic Coincidence Detectors for Precise Analysis of Cell Populations in Vivo. Glia 2011, 59, S100-S100.
18. Feil, S.; Valtcheva, N.; Feil, R., Inducible Cre mice. Methods Mol Blot 2009, 530, 343-63.
19. Sinha, D. K.; et al., Photocontrol of protein activity in cultured cells and zebrafish with one- and two-photon illumination. Chembiochem 2010, 11 (5), 653-63.
20. Casanova, E.; et al., ER-based double iCre fusion protein allows partial recombination in forebrain. Genesis 2002, 34 (3), 208-14.
21. Drent, E., et al., Pre-clinical evaluation of CD38 chimeric antigen receptor engineered T cells for the treatment of multiple myeloma. Haematologica 2016, 101 (5), 616-25.
22. Phototoxicity revisited. Nat Methods 2018, 15 (10), 751.

A number of embodiments of the invention have been described. Nevertheless, it can be understood that various modifications may be made without departing from the spirit and scope of the invention. Accordingly, other embodiments are within the scope of the following claims.

1. A method for remotely-controlling and non-invasively manipulating expression of an exogenous nucleic acid in a cell, or an immune cell,
the method comprising:
(a) inserting or expressing in a recombinantly engineered cell:
(i) a tamoxifen-gated photoactivatable split-Cre recombinase (TamPA-Cre) optogenetic system comprising:
(1) a nucleic acid encoding a cytosol-localizing mutant estrogen receptor ligand binding domain ERT2 fused to a N-terminal half of split Cre(2-59aa)-nMag (CreN-nMag) encoding segment, and,
(2) a nucleic acid encoding an NLS-pMag-CreC protein,
wherein an expressed NLS-pMag-CreC protein is nucleus-localized and the ERT2-CreN-nMag protein is cytosolically localized, and
(ii) a floxed exogenous nucleic acid operatively linked to a transcriptional regulatory element, optionally a constitutive or an inducible promoter, whose expression can be activated by an active Cre-lox site recombination event;
(b) administering or contacting the cell with tamoxifen or 4-hydroxytamoxifen (4-OHT), wherein a tamoxifen metabolite 4-hydroxytamoxifen (4-OHT) binds with the ERT2-CreN-nMag cytosolically localized protein to drive its nuclear localization to prime Tam PA-Cre; and
(c) exposing the cell to blue light to drive nMag-pMag heterodimerization, which restores active TamPA-Cre recombinase activity within the cell nucleus, thereby allowing expression of the floxed exogenous nucleic acid.

2. The method of claim 1, wherein the recombinantly engineered cell is administered in vivo,
wherein optionally the recombinantly engineered cell is administered to an individual in need thereof in vivo,
and optionally the blue light is administered to only a desired area or location in the individual in need thereof,
and optionally the desired area or location in the individual in need thereof is a site of a tumor or a growth,
and optionally the recombinantly engineered cell is injected into and/or adjacent or approximate to a cancer of a site of a tumor or a growth.

3. The method of claim 1, wherein the expressing of the floxed exogenous nucleic acid in the cell adds a function to the cell, or immune cell, or manipulates a physiologic and/or a genetic process in the cell, or immune cell, and optionally when the upregulated nucleic acid is a nucleic acid expressing (encoding) a CAR, a single chain antibody, or a single-domain antibody (also known as sdAb or nanobody) or an antibody fragment consisting of a single monomeric variable antibody domain, thereby adding a new specificity, function or target cell to a cell, an immune cell or a T cell.

4. The method of claim 1, wherein the cell is a human cell or a mammalian cell, or is a recombinantly engineered cell engineered to be transplanted or inserted into a tissue, an organ, an organism or an individual, or is or comprises a non-human transgenic animal genetically engineered to contain one or a plurality of recombinantly engineered cells.

5. The method of claim 1, wherein the tamoxifen is administered to the individual in need thereof by oral or topical administration, and optionally the tamoxifen is formulated as tamoxifen citrate,
and optionally the tamoxifen is formulated as a liquid, a gel or a solid, and optionally the liquid is formulated at about 10 mg/5 mL tamoxifen, and optionally the solid is a pill, a tablet, a geltab, a nanoparticle or a capsule, and optionally each solid formulation comprises about 15.2 mg of tamoxifen citrate which is equivalent to about 10 mg of tamoxifen, or each solid formulation comprises about 30.4 mg of tamoxifen citrate which is equivalent to about 20 mg of tamoxifen, and optionally the nanoparticle is a polylactide-co-glycolide (PLGA) nanoparticle loaded with tamoxifen or tamoxifen citrate[11].

6. The method of claim 1, wherein the cell or the individual in need thereof is first exposed to or administered tamoxifen or 4-hydroxytamoxifen (4-OHT) followed by being exposed to or administered a continuous or pulsed blue light,
wherein optionally the cells are exposed to between about 400 to 600 nM 4-hydroxytamoxifen (4-OHT), or about 500 nM 4-OHT,
and optionally blue light is applied to the cells between about 2 to 5 hours, or about 3 hours, following an initial exposure to tamoxifen,
and optionally the blue light frequency is about 400 to 500 nM,
and optionally the blue light is applied in a pulsed manner at about 1 second on to about 59 seconds off, or at about 5 seconds on to about 55 seconds off, optionally repeated over a time period of between about 1 hours and 36 hours, or between about 12 hours and 24 hours,
and optionally the blue light is continuously applied to the cells for between about 1 hour and 24 hours, or between about 2 hours and 12 hours.

7. The method of claim 1, wherein a chimeric antigen receptor (CAR) is expressed on a T cell surface after exposure of the T cell to tamoxifen followed by blue light, thereby activating the T cell to attack and/or kill a cancerous tissue, a cancer cell or a tumor cell,
wherein optionally the cancerous tissue, cancer cell or tumor cell is a local or skin or mucosal metastatic head/neck cancer, a melanoma, or a skin cancer or a skin growth.

8. The method of claim 1, wherein the cell is inside the body of an animal or a human in need thereof, and the recombinantly engineered cell is focused on or approximate to a tumor or a dysplastic or dysfunctional tissue.

9. The method of claim 1, wherein the method is used for the manipulation or correction of a pathological process, optionally, for eradicating a tumor or a cancer in an individual in vivo, wherein optionally the individual is a human or an animal.

10-12. (canceled)

13. A kit or formulation comprising a genetically engineered cell engineered to comprise:
(a) a tamoxifen-gated photoactivatable split-Cre recombinase (Tam PA-Cre) optogenetic system comprising:
(1) a nucleic acid encoding a cytosol-localizing mutant estrogen receptor ligand binding domain ERT2 fused to a N-terminal half of split Cre(2-59aa)-nMag (CreN-nMag) encoding segment, and,
(2) a nucleic acid encoding an NLS-pMag-CreC protein,
wherein an expressed NLS-pMag-CreC protein is nucleus-localized and the ERT2-CreN-nMaq protein is cytosolically localized, and
(b) a floxed exogenous nucleic acid operatively linked to a transcriptional regulatory element, optionally a constitutive or an inducible promoter, whose expression can be activated by an active Cre-lox site recombination event.

14. The method of claim 1, wherein the method further comprises modifying or adding a target capability or a function to the cell, or immune cell.

15. The method of claim 1, wherein the immune cell is a T cell, a primary T cell, a B cell, a monocyte, a macrophage, a dendritic cell or a natural killer cell.

16. The method of claim 1, wherein the exogenous nucleic acid is contained in a vector or expression cassette.

17. The method of claim 1, wherein the exogenous nucleic acid comprises a nucleic acid encoding (expressing) a protein.

18. The method of claim 17, wherein the protein is a therapeutic protein, or a transcriptional or translational regulatory protein, or a receptor, or a recombinant or an artificial T cell receptor (also known as a chimeric T cell receptor, a chimeric immunoreceptor, a chimeric antigen receptor (CAR), an antibody, a single chain antibody, or a single-domain antibody (also known as sdAb or nanobody) or an antibody fragment consisting of a single monomeric variable antibody domain.

19. The method of claim 1, wherein the recombinantly engineered cell is an immune cell or comprises a plurality of cells or immune cells.

20. The method of claim 1, wherein the lox site is or comprises a lox P site, a lox H site, a lox 511 site, a lox 5171 site, a lox 66 site, a lox 71 site, or equivalent lox sites.

21. The method of claim 1, wherein the constitutive promoter comprises an EF-1 alpha, PGK, CMV, CAG, SFFV, SV40 or equivalent constitutive promoter.

22. The method of claim 1, wherein the TamPA-Cre) optogenetic system is stably integrated into the genome of the cell or is episomally expressed or is contained in a non-integrated vector in the cell.

23. The method of claim 1, wherein the tamoxifen-gated photoactivatable split-Cre recombinase (Tam PA-Cre) optogenetic system and/or the floxed exogenous nucleic acid is contained in a lentivirus vector.
